(19) United States
(12) Patent Application Publication
(10) Pub. No.: US 2012/0088800 A1
(43) Pub. Date: Apr. 12, 2012

(54) 2-(SUBSTITUTED) (ARYLMETHYL, ARYLOXY and ARYLTHIO))-N-(SUBSTITUTED PYRIDIN-2-YL)-2-(SUBSTITUTED ARYL) COMPOUNDS AS SUBTYPE-SELECTIVE MODULATORS OF SPHINGOSINE-1-PHOSPHATE-3 (S1P3) RECEPTORS

(75) Inventors: Wenkui K. Fang, Irvine, CA (US); Ken Chow, Newport Coast, CA (US)

(73) Assignee: ALLERGAN, INC., Irvine, CA (US)

(21) Appl. No.: 13/320,374

(22) PCT Filed: Jun. 25, 2010
(86) PCT No.: PCT/US10/40014

§ 371 Date: Dec. 14, 2011

Related U.S. Application Data
(60) Provisional application No. 61/221,687, filed on Jun. 30, 2009.

(51) Int. Cl. A61K 31/44; A61P 27/06; A61P 9/00; C07D 213/84; A61P 27/02; C07D 213/75; A61P 7/10

(52) U.S. Cl. 514/344; 546/309; 514/352; 546/289

(57) ABSTRACT

The invention provides compounds represented by the formula (I), each of which compounds may have sphingosine-1-phosphate receptor agonist and or antagonist biological activity: wherein: A, m, n, p, a, X, Y and Z are defined in the specification. These compounds are useful for treating a disease or condition selected from the group consisting of glaucoma, dry eye, angiogenesis and pulmonary edema.

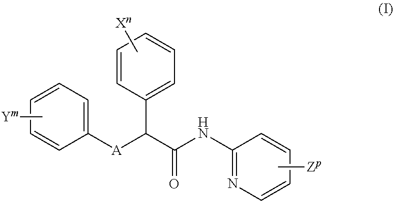

### CROSS REFERENCE

This application claims the benefit of U.S. Provisional Patent Application Ser. No. 61/221,687, filed on Jun. 30, 2009, the entire disclosure of which is incorporated herein by this specific reference.”

### FIELD OF THE INVENTION

The present invention relates to derivatives and/or analogues of sphingosine which are useful as drugs for the treatment of eye diseases and conditions selected from the group consisting of glaucoma, dry eye and angiogenesis and pulmonary edema.

### SUMMARY OF ART

Sphingosine is a compound having the chemical structure shown in the general formula described below, in which Y¹ is hydrogen. It is known that various sphingolipids, having sphingosine as a constituent, are widely distributed in the living body including on the surface of cell membranes of cells in the nervous system

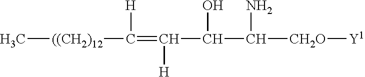

A sphingolipid is one of the lipids having important roles in the living body. A disease called lipidosis is caused by accumulation of a specified sphingolipid in the body. Sphingolipids present on cell membranes function to regulate cell growth; participate in the development and differentiation of cells; function in nerves; are involved in the infection and malignancy of cells; etc. Many of the physiological roles of sphingolipids remain to be solved. Recently the possibility that ceramide, a derivative of sphingosine, has an important role in the mechanism of cell signal transduction has been indicated, and studies about its effect on apoptosis and cell cycle have been reported

Sphingosine-1-phosphate is an important cellular metabolite, derived from ceramide that is synthesized de novo or as part of the sphingomeyeline cycle (in animals cells). It has also been found in insects, yeasts and plants.

The enzyme, ceramidase, acts upon ceramides to release sphingosine, which is phosphorylated by spingosine kinase, a ubiquitous enzyme in the cytosol and endoplasmic reticulum, to form sphingosine-1-phosphate. The reverse reaction can occur also by the action of sphingosine phosphatases, and the enzymes act in concert to control the cellular concentrations of the metabolite, which concentrations are always low. In plasma, such concentration can reach 0.2 to 0.9 μM, and the metabolite is found in association with the lipoproteins, especially the HDL. It should also be noted that sphingosine-1-phosphate formation is an essential step in the catabolism of sphingoid bases.

Like its precursors, sphingosine-1-phosphate is a potent messenger molecule that perhaps uniquely operates both intra- and inter-cellularly, but with very different functions from ceramides and sphingosine. The balance between these various sphingolipid metabolites may be important for health. For example, within the cell, sphingosine-1-phosphate promotes cellular division (mitosis) as opposed to cell death (apoptosis), which it inhibits. Intracellularly, it also functions to regulate calcium mobilization and cell growth in response to a variety of extracellular stimuli. Current opinion appears to suggest that the balance between sphingosine-1-phosphate and ceramide and/or spingosine levels in cells is critical for their viability. In common with the lysophospholipids, especially lysophosphatidic acid, with which it has some structural similarities, sphingosine-1-phosphate exerts many of its extra-cellular effects through interaction with five specific G protein-coupled receptors on cell surfaces. These are important for the growth of new blood vessels, vascular maturation, cardiac development and immunity, and for directed cell movement.

Sphingosine-1 phosphate is stored in relatively high concentrations in human platelets, which lack the enzymes responsible for its catabolism, and it is released into the blood stream upon activation of physiological stimuli, such as growth factors, cytokines, and receptor agonists and antigens. It may also have a critical role in platelet aggregation and thrombosis and could aggravate cardiovascular disease. On the other hand the relatively high concentration of the metabolite in high-density lipoproteins (HDL) may have beneficial implications for atherogenesis. For example, there are recent suggestions that sphingosine-1-phosphate, together with other lysolipids such as sphingosylphosphorylcholine and lysosulfatide, are responsible for the beneficial clinical effects of HDL by stimulating the production of the potent antiatherogenic signaling molecule nitric oxide by the vascular endothelium. In addition, like lysophosphatidic acid, it is a marker for certain types of cancer, and there is evidence that its role in cell division or proliferation may have an influence on the development of cancers. These are currently topics that are attracting great interest amongst medical researchers, and the potential for therapeutic intervention in sphingosine-1-phosphate metabolism is under active investigation.

Fungi and plants have sphingolipids and the major sphingosine contained in these organisms has the formula described below. It is known that these lipids have important roles in the cell growth of fungi and plants, but details of the roles remain to be solved.

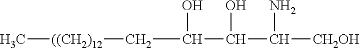

Recently it has been known that derivatives of sphingolipids and their related compounds exhibit a variety of biological activities through inhibition or stimulation of the metabolism pathways. These compounds include inhibitors of protein kinase C, inducers of apoptosis, immuno-suppressive compounds, antifungal compounds, and the like. Substances having these biological activities are expected to be useful compounds for various diseases.

Derivatives of sphingosine have been prepared in various patents. For example, see U.S. Pat. Nos. 4,952,683; 5,110,987; 6,235,912 and 6,239,297 which are hereby incorporated by reference.

Also, compounds which are similar to certain spingosine derivatives, but which are not reported as being ligands for the spingosine receptors are reported in various patents and published patent applications. See for example, U.S. Pat. Nos. 5,294,722; 5,102,901; 5,403,851, 5,580,878 and U.S. Patent Application Publication No. 2003/0125371 A2 which are hereby incorporated by reference in their entirety.

### SUMMARY OF THE INVENTION

The present invention provides compounds that are able to regulate the functions of sphingolipid, and pharmaceutical compositions comprising said compounds.

In one aspect of the present invention there is disclosed compounds, having sphingosine-1-phosphate receptor agonist and or antagonist biological activity, represented by the formula I:

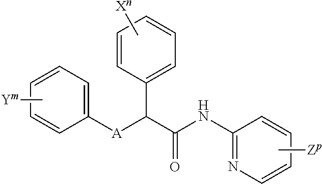

wherein:
   A is O, S or (CR₂)ₐ wherein R is selected from the group consisting of H, or lower alkyl;
   m, n and p are 0 or an integer of from 1 to 5, e.g. 1 or 2;
   a is 0 or 1,
   wherein when a is 1,
   X and Y are independently selected from the group consisting of alkyl, preferably lower alkyl, e.g. methyl, alkyloxy, preferably lower alkyloxy, e.g. methoxy, hydroxyl, halogen, e.g. chloro or bromo, nitrile, trifluoromethy and carboxy; and
   Z is selected from the group consisting of alkyl, preferably lower alkyl, e.g. methyl, alkyloxy, preferably lower alkoxy, e.g. methoxy, hydroxyl, halogen, e.g. fluoro, chloro, or bromo, nitrile, trifluoromethyl and carboxy and
   when a is 0,
   X, Y are independently selected from the group consisting of alkyl, preferably lower alkyl, e.g. methyl, alkyloxy, preferably lower alkoxy, e.g. methoxy, hydroxyl, halogen, nitrile, trifluoromethyl, and carboxy; and

Z is selected from the group consisting of alkyl, preferably lower alkyl, e.g. methyl. alkyloxy, preferably lower alkyloxy, e.g. methoxy, hydroxyl, halogen, preferably fluoro, nitrile, trifluoromethyl and carboxy provided however that when a is 0 and the pyridyl ring is (a) 5-chloro pyridyl, then both (i) m and n are not 0 and (ii) X and Y are not chloro or methyl or (b) 5-bromo or iodo pyridyl, then both m and n are not 0, or a pharmaceutically acceptable salt of said compound.

In another aspect of this invention, there is disclosed a method of treating or preventing a disease or condition selected from the group consisting of glaucoma, dry eye, angiogenesis and pulmonary edema, which comprises administering to a patient in need thereof a compound represented by the formula II

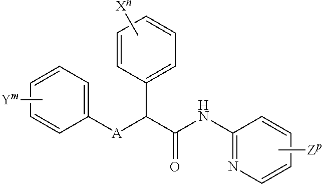

wherein:

A is O, S or (CR₂)ₐ wherein R is selected from the group consisting of H, or lower alkyl;

m, n and p are 0 or an integer of from 1 to 5, e.g. 1 or 2;

a is 0 or 1,

X and Y are independently selected from the group consisting of alkyl, preferably lower alkyl, e.g. methyl, alkyloxy, preferably lower alkyloxy, e.g. methoxy, hydroxyl, halogen, e.g. chloro or bromo, nitrile, trifluoromethy and carboxy; and.

Z is selected from the group consisting of alkyl, preferably lower alkyl, e.g. methyl, alkyloxy, preferably lower alkoxy, e.g. methoxy, hydroxyl, halogen, e.g. fluoro, chloro, or bromo, nitrile, trifluoromethyl and carboxy, or a pharmaceutically acceptable salt of said compound

### DETAILED DESCRIPTION OF THE INVENTION

Novel compounds having this general structure were synthesized and tested for sphingosine 1-phosphate receptor (S1P) activity using the FLIPR assay. Cells expressing the receptor of interest (S1P₁, S1P₂ or S1P₃) and a G-protein (Gqi5 or G16) are loaded with fluo-4, a calcium sensitive dye. After removal of excess dye by washing, the cells are placed in the FLIPR TETRA instrument. Baseline fluorescence readings are taken prior to addition the compound to be tested. Agonists will trigger the receptor to interact with the G-protein, leading to an increase in intracellular calcium. The increase in intracellular calcium causes an increase in the fluorescence of the cells, due to the presence of fluo-4. This fluorescence increase is recorded by the FLIPR TETRA. After the calcium transient signal has decreased towards baseline, the standard agonist sphingosine 1-phosphate is added. If the test compound is an antagonist, an initial calcium signal will not be generated and the antagonist will prevent the generation of a calcium signal from sphingosine 1-phosphate. The level of fluorescence is compared to that of sphingosine 1-phosphate, and the EC50 or IC50 of the compound determined by curve fitting.

The compounds in this invention will be useful for the treatment of mammals, including humans, for diseases or conditions selected from the group consisting of glaucoma, dry eye and angiogenesis disorders and pulmonary edema.

Specific Examples of the compounds of formula I include the compounds of Table 1, below.

TABLE 1

| Example | |
|---|---|
| Number | Structure |
| 1 | 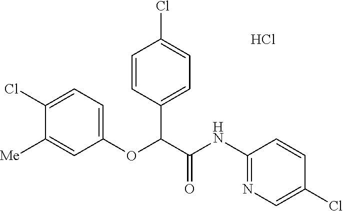 |
| 2 | 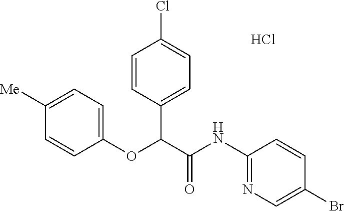 |
| 3 | 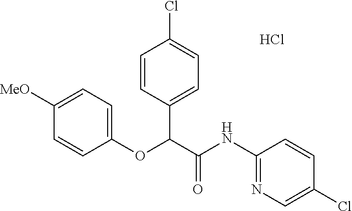 |
| 4 | 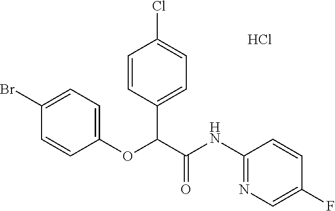 |
| 5 | 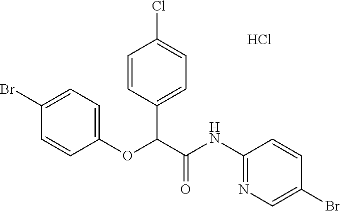 |
| 6 | 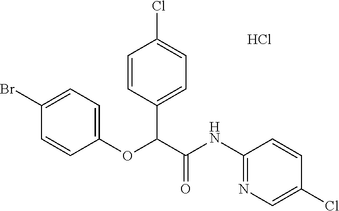 |
| 7 | 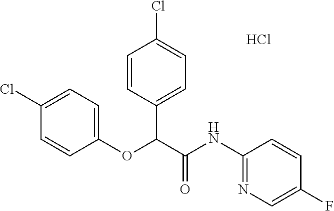 |
| 8 | 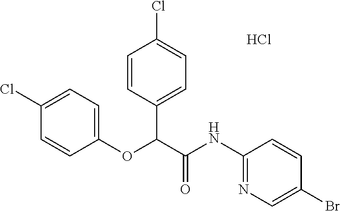 |
| 9 | 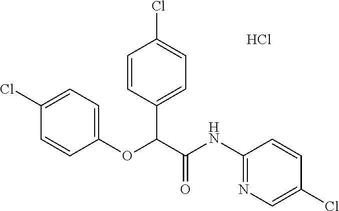 |
| 10 | 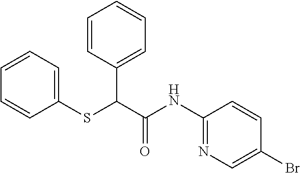 |
| 11 | 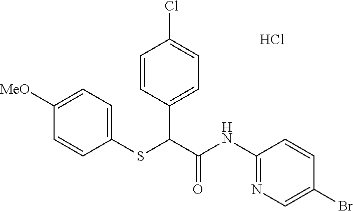 |
| 12 | 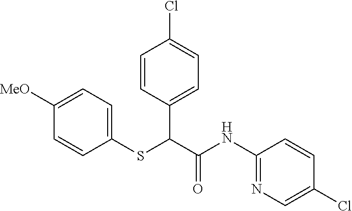 |
| 13 | 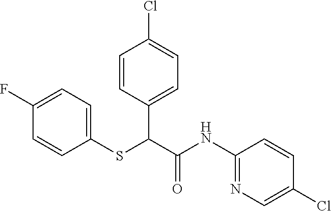 |
| 14 | 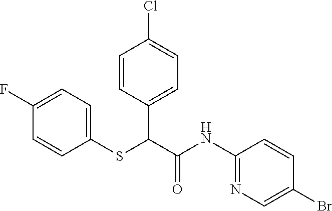 |
| 15 | 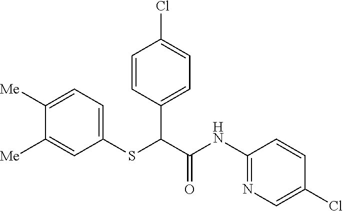 |
| 16 | 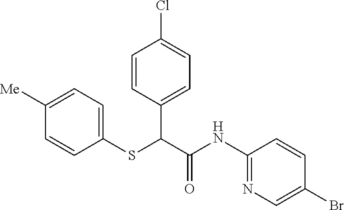 |
| 17 | 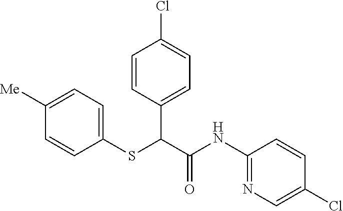 |
| 18 | 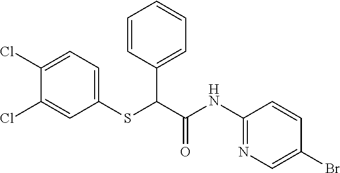 |
| 19 | 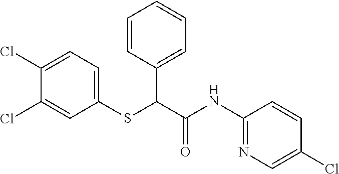 |
| 20 | 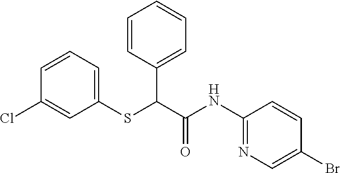 |
| 21 | 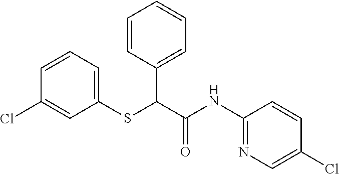 |
| 22 | 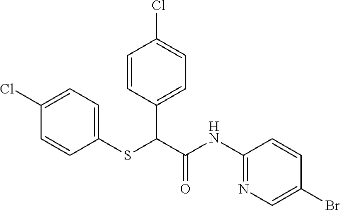 |
| 23 | 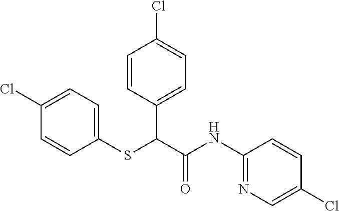 |
| 24 | 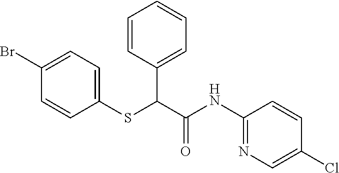 |
| 25 | 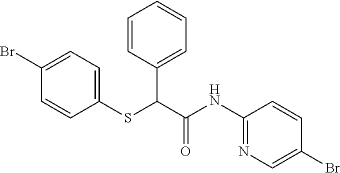 |
| 26 | 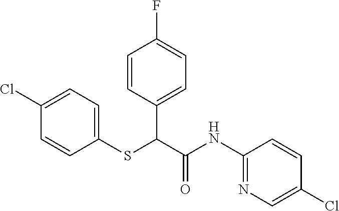 |
| 27 | 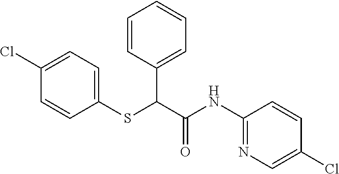 |
| 28 | 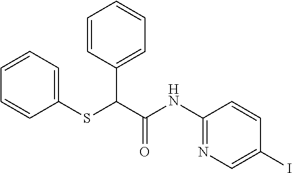 |
| 29 | 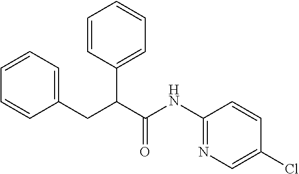 |
| 30 | 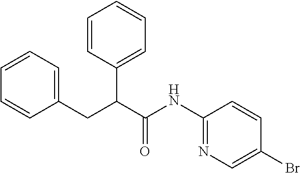 |
| 31 | 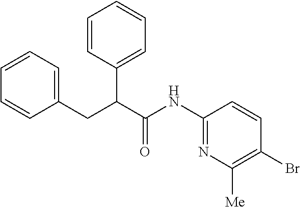 |
| 32 | 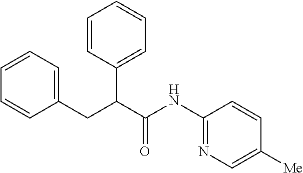 |
| 33 | 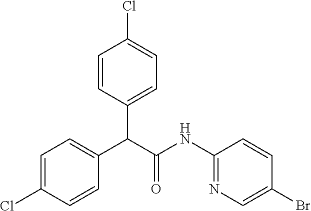 |
| 34 | 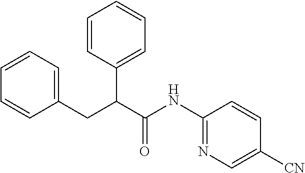 |

Some compounds within the scope of the invention may be prepared as depicted in the procedures described below, wherein R₁═X, R₂═Y and R₃═Z.

The invention is further illustrated by the following examples which are illustrative of a specific mode of practicing the invention and are not intended as limiting the scope of the claims.

### General Procedure A for the Synthesis of 2-(Substituted Phenoxy)-2-(substituted phenyl)-N-(substituted pyridin-2-yl)acetamide Hydrochloride

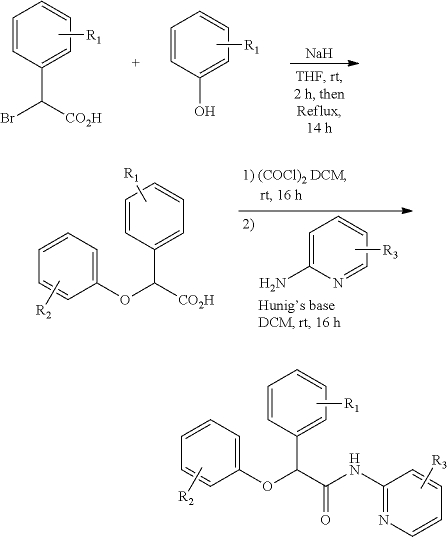

The intermediate 2-(substituted phenoxy)-2-(substituted phenyl)acetic acid is obtained from the appropriate substituted α-bromophenylacetic acid (1 eq), substituted phenol (1 eq) and sodium hydride (2-3 eq) according to the synthetic protocol published in William T. Brady, Yun Seng F. Giang, Alan P. Marchand, An Hsiang Wu J. Org. Chem.; 1987; 52(15); 3457-3461, (hereinafter Brady et al.).

This carboxylic acid is then converted into the correspond acid chloride by the method published in Martin Newcomb, Michael T. Burchill, Thomas M. Deeb J. Am. Chem. Soc.; 1988; 110(19); 6528-6535 (hereinafter Newcomb et al.). The acid chloride (1 eq) and an appropriate substituted 2-aminopyridine (1.5-2.0 eq) are dissolved in 50 ml of dichloromethane at room temperature. Hünig's base (3.50 mL) is added, and the resulting reaction mixture is stirred at room temperature for 14 hours. The reaction mixture is then concentrated and diluted with ethyl acetate. The organic phase is washed sequentially with water and brine, then dried with sodium sulfate and concentrated. Flash chromatography (30% EtOAc/hexanes) gives the desired free base, which is mixed with HCl in MeOH. Concentration and recrystallization from ether gives the final desired product in HCl salt form.

### General Procedure B for the Synthesis of 2-(Substituted phenoxy)-2-(substituted phenyl)-N-(substituted pyridin-2-yl)acetamide Hydrochloride

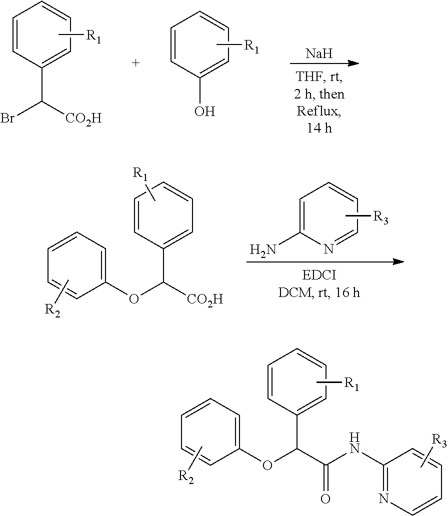

The intermediate 2-(substituted phenoxy)-2-(substituted phenyl)acetic acid is obtained from the appropriate substituted α-bromophenylacetic acid (1 eq), substituted phenol (1 eq) and sodium hydride (2-3 eq) according to the synthetic protocol published in William T. Brady, Yun Seng F. Clang, Alan P. Marchand, An Hsiang Wu J. Org. Chem.; 1987; 52(15); 3457-3461. This carboxylic acid is then mixed with an appropriate substituted 2-aminopyridine (1.2-1.5 eq) in 50 ml of dichloromethane at room temperature. EDCI (1.5 to 2 eq) is added, followed by a catalytic amount of DMAP (30 mg). The resulting reaction mixture is stirred at room temperature for 14 hours. The reaction mixture is then concentrated and chromatographed (30% EtOAc/hexanes) to give the desired free base, which is mixed with HCl in MeOH. Concentration and recrystallization from ether gives the final desired product in HCl salt form.

### Example 1

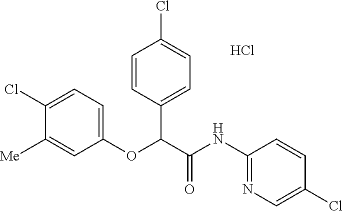

### Synthesis of 2-(4-chloro-3-methylphenoxy)-2-(4-chlorophenyl)-N-(5-chloropyridin-2-yl)acetamide hydrochloride

The title compound was generated from commercially available 2-bromo-2-(4-chlorophenyl)acetic acid, 4-chloro-3-methylphenol and 5-chloropyridin-2-amine according to General procedure B described above. The intermediate 2-(4-chloro-3-methylphenoxy)-2-(4-chlorophenyl)acetic acid was used in the subsequent synthetic transformation without further purification.

2-(4-Chloro-3-methylphenoxy)-2-(4-chlorophenyl)acetic acid: The title compound was obtained from commercially available 2-bromo-2-(4-chlorophenyl)acetic acid (12.00 g, 48.10 mmol), 4-chloro-3-methylphenol (5.90 g, 41.38 mmol) and sodium hydride (60% oil dispersion, 5.50 g, 137.50 mmol) according to the protocols as outlined in general procedure B above. This acid was used in the next synthetic step without further purification.

2-(4-Chloro-3-methylphenoxy)-2-(4-chlorophenyl)-N-(5-chloropyridin-2-yl)acetamide hydrochloride: The title compound was obtained from 2-(4-chloro-3-methylphenoxy)-2-(4-chlorophenyl)acetic acid (taken from the previous step, crude, 3.00 g, 9.64 mmol), 5-chloropyridin-2-amine (1.50 g, 11.67 mmol) and EDCI (3.50 g, 18.25 mmol) according to the protocols as outlined in general procedure B above. Spectroscopic Data: ¹H NMR (500 MHz, DMSO-d₆) δ ppm 2.23 (s, 3H), 6.21 (s, 1H), 6.85 (dd, J=8.80, 2.93 Hz, 1H), 7.04 (d, J=2.69 Hz, 1H), 7.29 (d, J=8.56 Hz, 1H), 7.46 (d, J=8.56 Hz, 2H), 7.67 (d, J=8.56 Hz, 2H), 7.85 (dd, J=8.93, 2.57 Hz, 1H), 7.97 (d, J=8.80 Hz, 1H), 8.36 (d, J=2.44 Hz, 1H), 11.28 (s, 1H), 11.43-11.93 (br s, 1H).

### Example 2

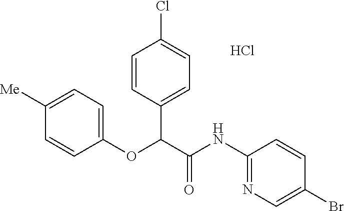

### Synthesis of N-(5-bromopyridin-2-yl)-2-(4-chlorophenyl)-2-(p-tolyloxy)acetamide hydrochloride

The title compound was generated from commercially available 2-bromo-2-(4-chlorophenyl)acetic acid, p-cresol and 5-bromopyridin-2-amine according to General procedure B described above. The intermediate 2-(4-chlorophenyl)-2-(p-tolyloxy)acetic acid was used in the subsequent synthetic transformation without further purification.

2-(4-Chloro-phenyl)-2-(p-tolyloxy)acetic acid: The title compound was obtained from commercially available 2-bromo-2-(4-chlorophenyl)acetic acid (12.00 g, 48.10 mmol), p-cresol (5.20 g, 48.09 mmol) and sodium hydride (60% oil dispersion, 5.50 g, 137.50 mmol) according to the protocols as outlined in general procedure B above. This acid was used in the next synthetic step without further purification.

N-(5-Bromopyridin-2-yl)-2-(4-chlorophenyl)-2-(p-tolyloxy)acetamide hydrochloride: The title compound was obtained from 2-(4-chloro-phenyl)-2-(p-tolyloxy)acetic acid (taken from the previous step, crude, 2.70 g, 9.76 mmol), 5-bromopyridin-2-amine (1.90 g, 10.98 mmol) and EDCI (3.50 g, 18.25 mmol) according to the protocols as outlined in general procedure B above. Spectroscopic Data: ¹H NMR (500 MHz, DMSO-d₆) δ ppm 2.18 (s, 3H), 6.09 (s, 1H), 6.89 (d, J=8.56 Hz, 2H), 7.07 (d, J=8.80 Hz, 2H), 7.47 (d, J=8.56 Hz, 2H), 7.66 (d, J=8.56 Hz, 2H), 7.90-7.95 (m, 1H), 7.96-8.01 (m, 1H), 8.05 (br s, 1H) 8.45 (d, J=2.45 Hz, 1H), 11.17 (s, 1H).

### Example 3

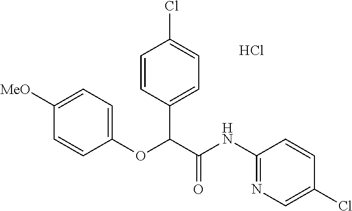

### Synthesis of 2-(4-chlorophenyl)-N-(5-chloropyridin-2-yl)-2-(4-methoxyphenoxy)acetamide hydrochloride

The title compound was generated from commercially available 2-bromo-2-(4-chlorophenyl)acetic acid, 4-methoxyphenol and 5-chloropyridin-2-amine according to General procedure B described above. The intermediate 2-(4-chlorophenyl)-2-(4-methoxyphenoxy)acetic acid was used in the subsequent synthetic transformation without further purification.

2-(4-Chlorophenyl)-2-(4-methoxyphenoxy)acetic acid: The title compound was obtained from commercially available 2-bromo-2-(4-chlorophenyl)acetic acid (12.00 g, 48.10 mmol), 4-methoxyphenol (6.00 g, 48.33 mmol) and sodium hydride (60% oil dispersion, 5.50 g, 137.50 mmol) according to the protocols as outlined in general procedure B above. This acid was used in the next synthetic step without further purification.

2-(4-Chlorophenyl)-N-(5-chloropyridin-2-yl)-2-(4-methoxyphenoxy)acetamide hydrochloride: The title compound was obtained from 2-(4-chlorophenyl)-2-(4-methoxyphenoxy)acetic acid (taken from the previous step, crude, 2.90 g, 9.91 mmol), 5-chloropyridin-2-amine (1.50 g, 11.67 mmol) and EDCI (3.80 g, 19.82 mmol) according to the protocols as outlined in general procedure B above. Spectroscopic Data: ¹H NMR (500 MHz, DMSO-d₆) δ ppm 3.65 (s, 3H), 6.06 (s, 1H), 6.76-6.88 (m, 2H), 6.89-7.03 (m, 2 H), 7.38-7.53 (m, 2H), 7.66 (d, J=8.56 Hz, 2H), 7.87 (dd, J=8.93, 2.57 Hz, 1H), 7.99 (d, J=9.05 Hz, 1H), 8.38 (d, J=2.69 Hz, 1H), 10.60 (br s, 1H), 11.16 (s, 1H).

### Example 4

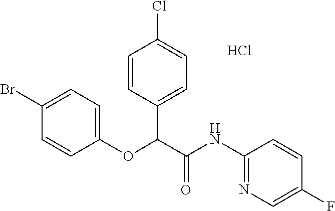

### Synthesis of 2-(4-bromophenoxy)-2-(4-chlorophenyl)-N-(5-fluoropyridin-2-yl)acetamide hydrochloride

The title compound was generated from commercially available 2-bromo-2-(4-chlorophenyl)acetic acid, 4-bromophenol and 5-fluoropyridin-2-amine according to General procedure B described above. The intermediate 2-(4-bromophenoxy)-2-(4-chlorophenyl)acetic acid was used in the subsequent synthetic transformation without further purification.

2-(4-Bromophenoxy)-2-(4-chlorophenyl)acetic acid: The title compound was obtained from commercially available 2-bromo-2-(4-chlorophenyl)acetic acid (12.00 g, 48.10 mmol), 4-bromophenol (8.30 g, 47.97 mmol) and sodium hydride (60% oil dispersion, 5.50 g, 137.50 mmol) according to the protocols as outlined in general procedure B above. This acid was used in the next synthetic step without further purification.

2-(4-Bromophenoxy)-2-(4-chlorophenyl)-N-(5-fluoropyridin-2-yl)acetamide hydrochloride: The title compound was obtained from 2-(4-bromophenoxy)-2-(4-chlorophenyl)acetic acid (taken from the previous step, crude, 3.40 g, 9.95 mmol), 5-fluoropyridin-2-amine (1.10 g, 9.81 mmol) and EDCI (3.50 g, 18.26 mmol) according to the protocols as outlined in general procedure B above. Spectroscopic Data: ¹H NMR (500 MHz, DMSO-d₆) δ ppm 6.22 (s, 1H), 6.81-7.15 (m, 2H), 7.45 (t, J=9.05 Hz, 4H), 7.68 (d, J=8.56 Hz, 3H), 7.98 (dd, J=9.05, 4.16 Hz, 1H), 8.32 (d, J=2.93 Hz, 1H), 10.88-11.21 (br s, 1H), 11.25 (s, 1H).

### Example 5

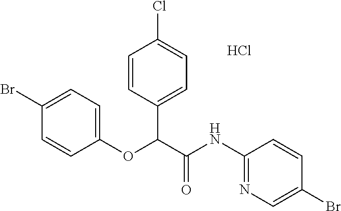

### Synthesis of 2-(4-bromophenoxy)-N-(5-bromopyridin-2-yl)-2-(4-chlorophenyl)acetamide hydrochloride

The title compound was generated from commercially available 2-bromo-2-(4-chlorophenyl)acetic acid, 4-bromophenol and 5-bromopyridin-2-amine according to General procedure B described above. The intermediate 2-(4-bromophenoxy)-2-(4-chlorophenyl)acetic acid was used in the subsequent synthetic transformation without further purification.

2-(4-Bromophenoxy)-2-(4-chlorophenyl)acetic acid: The title compound was obtained from commercially available 2-bromo-2-(4-chlorophenyl)acetic acid (12.00 g, 48.10 mmol), 4-bromophenol (8.30 g, 47.97 mmol) and sodium hydride (60% oil dispersion, 5.50 g, 137.50 mmol) according to the protocols as outlined in general procedure B above. This acid was used in the next synthetic step without further purification.

2-(4-Bromophenoxy)-N-(5-bromopyridin-2-yl)-2-(4-chlorophenyl)acetamide hydrochloride: The title compound was obtained from 2-(4-bromophenoxy)-2-(4-chlorophenyl)acetic acid (taken from the previous step, crude, 3.40 g, 9.95 mmol), 5-bromopyridin-2-amine (1.90 g, 10.98 mmol) and EDCI (3.50 g, 18.26 mmol) according to the protocols as outlined in general procedure B above. Spectroscopic Data: ¹H NMR (500 MHz, DMSO-d₆) δ ppm 6.22 (s, 1H), 6.97 (q, J=5.62 Hz, 2H), 7.45 (dd, J=10.51, 8.80 Hz, 4H), 7.67 (d, J=8.56 Hz, 2H), 7.84-8.06 (m, 2H), 8.43 (d, J=2.45 Hz, 1H), 11.31 (s, 1H), 11.59 (br s, 1H).

### Example 6

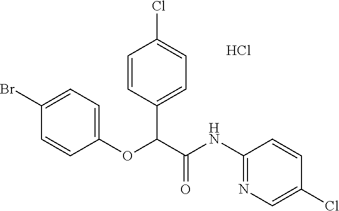

### Synthesis of 2-(4-bromophenoxy)-2-(4-chlorophenyl)-N-(5-chloropyridin-2-yl)acetamide hydrochloride

The title compound was generated from commercially available 2-bromo-2-(4-chlorophenyl)acetic acid, 4-bromophenol and 5-chloropyridin-2-amine according to General procedure B described above. The intermediate 2-(4-bromophenoxy)-2-(4-chlorophenyl)acetic acid was used in the subsequent synthetic transformation without further purification.

2-(4-Bromophenoxy)-2-(4-chlorophenyl)acetic acid: The title compound was obtained from commercially available 2-bromo-2-(4-chlorophenyl)acetic acid (12.00 g, 48.10 mmol), 4-bromophenol (8.30 g, 47.97 mmol) and sodium hydride (60% oil dispersion, 5.50 g, 137.50 mmol) according to the protocols as outlined in general procedure B above. This acid was used in the next synthetic step without further purification.

2-(4-Bromophenoxy)-2-(4-chlorophenyl)-N-(5-chloropyridin-2-yl)acetamide hydrochloride: The title compound was obtained from 2-(4-bromophenoxy)-2-(4-chlorophenyl)acetic acid (taken from the previous step, crude, 3.40 g, 9.95 mmol), 5-chloropyridin-2-amine (1.50 g, 11.67 mmol) and EDCI (3.80 g, 19.82 mmol) according to the protocols as outlined in general procedure B above. Spectroscopic Data: ¹H NMR (500 MHz, DMSO-d₆) δ ppm 6.22 (s, 1H), 6.97 (d, J=9.05 Hz, 2H), 7.46 (dd, J=9.78, 8.80 Hz, 4H), 7.67 (d, J=8.56 Hz, 2H), 7.85 (dd, J=9.05, 2.69 Hz, 1H), 7.97 (d, J=9.05 Hz, 1H), 8.36 (d, J=2.69 Hz, 1H), 10.07 (br s, 1H), 11.31 (s, 1H).

### Example 7

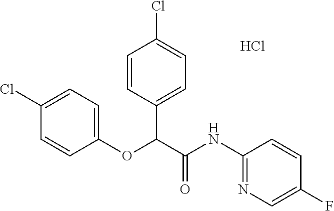

### Synthesis of 2-(4-chlorophenoxy)-2-(4-chlorophenyl)-N-(5-fluoropyridin-2-yl)acetamide hydrochloride

The title compound was generated from commercially available 2-bromo-2-(4-chlorophenyl)acetic acid, 4-chlorophenol, oxalyl chloride and 5-fluoropyridin-2-amine according to General procedure A described above. The intermediate 2-(4-chlorophenoxy)-2-(4-chlorophenyl)acetyl chloride was used in the subsequent synthetic transformation without further purification.

2-(4-Chlorophenoxy)-2-(4-chlorophenyl)acetyl chloride: The title compound was obtained from commercially available 2-bromo-2-(4-chlorophenyl)acetic acid (12.26 g, 49.14 mmol), 4-chlorophenol (6.30 g, 49.00 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

2-(4-Chlorophenoxy)-2-(4-chlorophenyl)-N-(5-fluoropyridin-2-yl)acetamide hydrochloride: The title compound was obtained from 2-(4-chlorophenoxy)-2-(4-chlorophenyl)acetic acid (taken from the previous step, crude, 2.00 g, 6.34 mmol), 5-fluoropyridin-2-amine (1.20 g, 10.70 mmol) and Hünig's base (3.50 mL, 20.09 mmol) according to the protocols as outlined in general procedure A above. Spectroscopic Data: ¹H NMR (500 MHz, DMSO-d₆) δ ppm 6.17 (s, 1H), 7.03 (d, J=8.80 Hz, 2H), 7.07 (dd, J=9.90, 4.52 Hz, 1H), 7.32-7.37 (m, 1H), 7.48 (d, J=8.56 Hz, 2H), 7.67 (d, J=8.31 Hz, 2H), 7.71 (td, J=8.68, 2.93 Hz, 1H), 8.00 (td, J=10.03, 7.34 Hz, 1H), 8.19 (t, J=3.06 Hz, 1H), 8.34 (d, J=3.18 Hz, 1H), 11.20 (s, 1H).

### Example 8

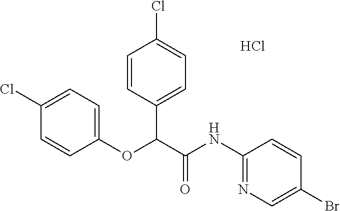

### Synthesis of N-(5-bromopyridin-2-yl)-2-(4-chlorophenoxy)-2-(4-chlorophenyl)acetamide hydrochloride

The title compound was generated from commercially available 2-bromo-2-(4-chlorophenyl)acetic acid, 4-chlorophenol, oxalyl chloride and 5-bromopyridin-2-amine according to General procedure A described above. The intermediate 2-(4-chlorophenoxy)-2-(4-chlorophenyl)acetyl chloride was used in the subsequent synthetic transformation without further purification.

2-(4-Chlorophenoxy)-2-(4-chlorophenyl)acetyl chloride: The title compound was obtained from commercially available 2-bromo-2-(4-chlorophenyl)acetic acid (12.26 g, 49.14 mmol), 4-chlorophenol (6.30 g, 49.00 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

N-(5-Bromopyridin-2-yl)-2-(4-chlorophenoxy)-2-(4-chlorophenyl)acetamide hydrochloride: The title compound was obtained from 2-(4-chlorophenoxy)-2-(4-chlorophenyl)acetic acid (taken from the previous step, crude, 3.15 g, 9.98 mmol), 5-bromopyridin-2-amine (1.80 g, 10.40 mmol) and Hünig's base (5.00 mL, 28.66 mmol) according to the protocols as outlined in general procedure A above. Spectroscopic Data: ¹H NMR (500 MHz, DMSO-d₆) δ ppm 6.16 (s, 1H), 7.02 (d, J=9.05 Hz, 2H), 7.34 (d, J=9.05 Hz, 2H), 7.48 (d, J=8.56 Hz, 2H), 7.66 (d, J=8.56 Hz, 2H), 7.89-7.95 (m, 1 H), 7.96-8.03 (m, 1H), 8.45 (d, J=2.69 Hz, 1H), 10.03-10.47 (br s, 1H), 11.25 (s, 1H).

### Example 9

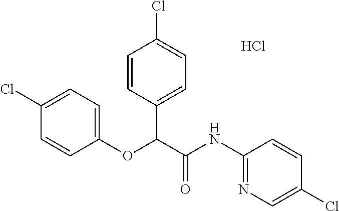

### Synthesis of 2-(4-chlorophenoxy)-2-(4-chlorophenyl)-N-(5-chloropyridin-2-yl)acetamide hydrochloride

The title compound was generated from commercially available 2-bromo-2-(4-chlorophenyl)acetic acid, 4-chlorophenol, oxalyl chloride and 5-chloropyridin-2-amine according to General procedure A described above. The intermediate 2-(4-chlorophenoxy)-2-(4-chlorophenyl)acetyl chloride was used in the subsequent synthetic transformation without further purification.

2-(4-Chlorophenoxy)-2-(4-chlorophenyl)acetyl chloride: The title compound was obtained from commercially available 2-bromo-2-(4-chlorophenyl)acetic acid (12.26 g, 49.14 mmol), 4-chlorophenol (6.30 g, 49.00 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

2-(4-Chlorophenoxy)-2-(4-chlorophenyl)-N-(5-chloropyridin-2-yl)acetamide hydrochloride: The title compound was obtained from 2-(4-chlorophenoxy)-2-(4-chlorophenyl)acetic acid (taken from the previous step, crude, 3.15 g, 9.98 mmol), 5-chloropyridin-2-amine (1.30 g, 10.11 mmol) and Hünig's base (5.00 mL, 28.66 mmol) according to the protocols as outlined in general procedure A above. Spectroscopic Data: ¹H NMR (500 MHz, DMSO-d₆) δ ppm 6.18 (s, 1H), 7.02 (d, J=9.05 Hz, 2H), 7.34 (d, J=9.05 Hz, 2H), 7.48 (d, J=8.31 Hz, 2H), 7.67 (d, J=8.56 Hz, 2H), 7.87 (dd, J=9.05, 2.69 Hz, 1H), 7.98 (d, J=9.05 Hz, 1H), 8.38 (d, J=2.69 Hz, 1H), 10.75 (br s, 1H), 11.28 (s, 1H).

### General Procedure A for the Synthesis of 2-(Substituted Phenylthio)-2-(substituted phenyl)-N-(substituted pyridin-2-yl)acetamide Hydrochloride

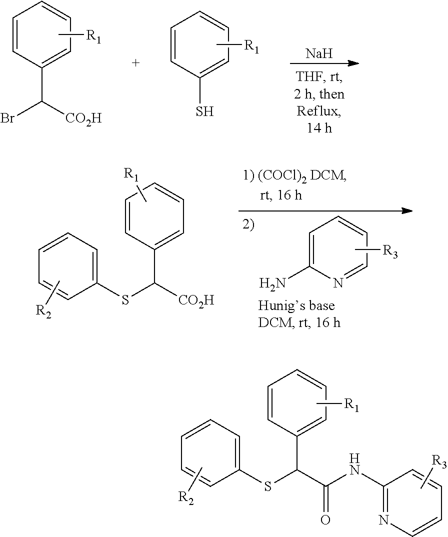

The intermediate 2-(substituted phenylthio)-2-(substituted phenyl)acetic acid was obtained from the appropriately substituted-bromophenylacetic acid (1 eq), substituted thiophenol (1 eq) and sodium hydride (2-3 eq) according to the synthetic protocol published in Brady et al. This carboxylic acid was then converted into the corresponding acid chloride by the method published in Newcomb et al. The acid chloride (1 eq) and an appropriately substituted 2-aminopyridine (1.5-2.0 eq) were dissolved in 50 ml of dichloromethane at room temperature. Hünig's base (3.50 mL) was added, and the resulting reaction mixture was stirred at room temperature for 14 hours. The reaction mixture was then concentrated and diluted with ethyl acetate. The organic phase was washed sequentially with water and brine, then dried with sodium sulfate and concentrated. Flash chromatography (30% EtOAc/hexanes) gave the desired free base, which was mixed with HCl in MeOH. Concentration and recrystallization from ether gave the final desired product in HCl salt form.

### General Procedure B for the Synthesis of 2-(Substituted phenylthio)-2-(substituted phenyl)-N-(substituted pyridin-2-yl)acetamide Hydrochloride

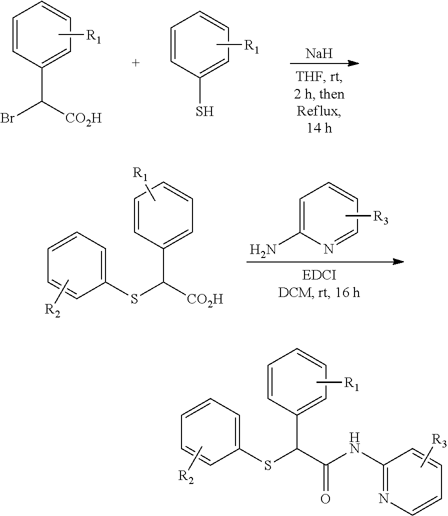

The intermediate 2-(substituted phenylthio)-2-(substituted phenyl)acetic acid was obtained from the appropriately substituted-bromophenylacetic acid (1 eq), substituted thiophenol (1 eq) and sodium hydride (2-3 eq) according to the synthetic protocol published in Brady et al. This carboxylic acid was then mixed with an appropriately substituted 2-aminopyridine (1.2-1.5 eq) in 50 ml of dichloromethane at room temperature. EDCI (1.5 to 2 eq) was added, followed by a catalytic amount of DMAP (30 mg). The resulting reaction mixture was stirred at room temperature for 14 hours. The reaction mixture was then concentrated and chromatographed (30% EtOAc/hexanes) to give the desired free base, which was mixed with HCl in MeOH. Concentration and recrystallization from ether gave the final desired product in HCl salt form.

### Example 10

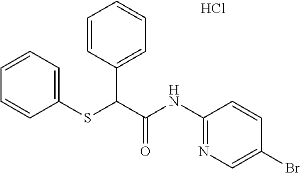

### Synthesis of N-(5-bromopyridin-2-yl)-2-phenyl-2-(phenylthio)acetamide hydrochloride

The title compound was generated from commercially available 2-phenyl-2-(phenylthio)acetic acid, oxalyl chloride and 5-chloropyridin-2-amine according to General procedure A described above.

N-(5-Bromopyridin-2-yl)-2-phenyl-2-(phenylthio)acetamide hydrochloride: The title compound was obtained from 2-phenyl-2-(phenylthio)acetic acid (commercially available from VWR), 5-bromoopyridin-2-amine (1.60 g, 9.25 mmol) according to the protocols as outlined in general procedure A above. Spectroscopic Data: ¹H NMR (500 MHz, DMSO-d₆) δ ppm 5.62 (s, 1H), 7.17-7.23 (m, 1H), 7.28 (t, J=7.58 Hz, 3H), 7.31-7.37 (m, 4H), 7.54 (d, J=7.09 Hz, 2H), 7.97 (d, J=1.47 Hz, 2H), 8.41 (t, J=1.59 Hz, 1H), 11.17 (s, 1H).

### Example 11

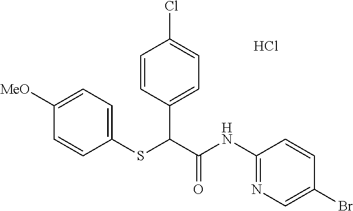

### Synthesis of N-(5-bromopyridin-2-yl)-2-(4-chlorophenyl)-2-(4-methoxyphenylthio)acetamide hydrochloride

The title compound was generated from commercially available 2-bromo-2-(4-chlorophenyl)acetic acid, 4-methoxybenzenethiol and 5-bromopyridin-2-amine according to general procedure B described above. The intermediate 2-(4-chlorophenyl)-2-(4-methoxyphenylthio)acetic acid was used in the subsequent synthetic transformation without further purification.

2-(4-Chlorophenyl)-2-(4-methoxyphenylthio)acetic acid: The title compound was obtained from commercially available 2-bromo-2-(4-chlorophenyl)acetic acid (10.61 g, 42.53 mmol), 4-methoxybenzenethiol (6.30 g, 44.94 mmol) and sodium hydride (60% oil dispersion, 5.50 g, 137.50 mmol) according to the protocols as outlined in general procedure B above. This acid was used in the next synthetic step without further purification.

N-(5-Bromopyridin-2-yl)-2-(4-chlorophenyl)-2-(4-methoxyphenylthio)acetamide hydrochloride: The title compound was obtained from 2-(4-chlorophenyl)-2-(4-methoxyphenylthio)acetic acid (taken from the previous step, crude, 3.00 g, 9.72 mmol), 5-bromopyridin-2-amine (1.90 g, 10.98 mmol) and EDCI (3.50 g, 18.25 mmol) according to the protocols as outlined in general procedure B above. Spectroscopic Data: ¹H NMR (500 MHz, DMSO-d₆) δ ppm 3.69 (s, 3H), 5.37 (s, 1H), 6.85 (q, J=5.22 Hz, 2H), 7.29 (d, J=8.80 Hz, 2H), 7.36-7.43 (m, 2H), 7.45-7.51 (m, 2H), 7.98 (d, J=1.47 Hz, 2H), 8.41 (t, J=1.59 Hz, 1H), 9.67 (br. s., 1H), 11.08 (s, 1H).

### Example 12

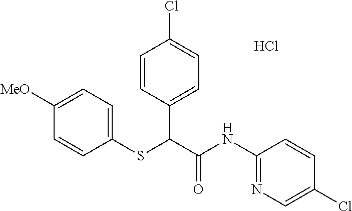

### Synthesis of 2-(4-chlorophenyl)-N-(5-chloropyridin-2-yl)-2-(4-methoxyphenylthio)acetamide hydrochloride

The title compound was generated from commercially available 2-bromo-2-(4-chlorophenyl)acetic acid, 4-methoxybenzenethiol and 5-chloropyridin-2-amine according to general procedure B described above. The intermediate 2-(4-chlorophenyl)-2-(4-methoxyphenylthio)acetic acid was used in the subsequent synthetic transformation without further purification.

2-(4-Chlorophenyl)-2-(4-methoxyphenylthio)acetic acid: The title compound was obtained from commercially available 2-bromo-2-(4-chlorophenyl)acetic acid (10.61 g, 42.53 mmol), 4-methoxybenzenethiol (6.30 g, 44.94 mmol) and sodium hydride (60% oil dispersion, 5.50 g, 137.50 mmol) according to the protocols as outlined in general procedure B above. This acid was used in the next synthetic step without further purification

2-(4-Chlorophenyl)-N-(5-chloropyridin-2-yl)-2-(4-methoxyphenylthio)acetamide hydrochloride: The title compound was obtained from 2-(4-chlorophenyl)-2-(4-methoxyphenylthio)acetic acid (taken from the previous step, crude, 3.00 g, 9.72 mmol), 5-chloropyridin-2-amine (1.50 g, 11.67 mmol) and EDCI (3.50 g, 18.25 mmol) according to the protocols as outlined in general procedure B above. Spectroscopic Data: ¹H NMR (500 MHz, DMSO-d₆) δ ppm 3.69 (s, 3H), 5.37 (s, 1H), 6.86 (q, J=5.30 Hz, 2H), 7.29 (q, J=5.14 Hz, 2H), 7.39 (q, J=4.40 Hz, 2H), 7.43-7.53 (m, 2H), 7.88 (dd, J=9.05, 2.69 Hz, 1H), 8.03 (d, J=8.80 Hz, 1H), 8.34 (d, J=2.69 Hz, 1H), 9.12 (br. s., 1H), 11.09 (s, 1H).

### Example 13

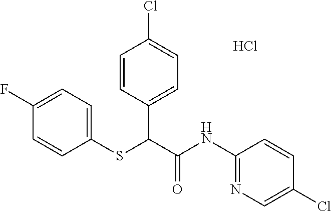

### Synthesis of 2-(4-chlorophenyl)-N-(5-chloropyridin-2-yl)-2-(4-fluorophenylthio)acetamide hydrochloride

The title compound was generated from commercially available 2-bromo-2-(4-chlorophenyl)acetic acid, 4-fluorobenzenethiol and 5-chloropyridin-2-amine according to general procedure B described above. The intermediate 2-(4-chlorophenyl)-2-(4-fluorophenylthio)acetic acid was used in the subsequent synthetic transformation without further purification.

2-(4-Chlorophenyl)-2-(4-fluorophenylthio)acetic acid: The title compound was obtained from commercially available 2-bromo-2-(4-chlorophenyl)acetic acid (10.45 g, 41.89 mmol), 4-fluorobenzenethiol (5.50 g, 42.91 mmol) and sodium hydride (60% oil dispersion, 5.50 g, 137.50 mmol) according to the protocols as outlined in general procedure B above. This acid was used in the next synthetic step without further purification.

2-(4-Chlorophenyl)-N-(5-chloropyridin-2-yl)-2-(4-fluorophenylthio)acetamide hydrochloride: The title compound was obtained from 2-(4-chlorophenyl)-2-(4-fluorophenylthio)acetic acid (taken from the previous step, crude, 3.00 g, 9.65 mmol), 5-chloropyridin-2-amine (1.50 g, 11.67 mmol) and EDCI (3.50 g, 18.26 mmol) according to the protocols as outlined in general procedure B above. Spectroscopic Data: ¹H NMR (500 MHz, DMSO-d₆) δ ppm 5.48 (s, 1H), 7.15 (t, J=8.80 Hz, 2H), 7.36-7.45 (m, 4H), 7.50 (d, J=8.31 Hz, 2H), 7.88 (dd, J=8.93, 2.57 Hz, 1H), 8.02 (d, J=8.80 Hz, 1H), 8.35 (d, J=2.45 Hz, 1H), 11.14 (s, 1H).

### Example 14

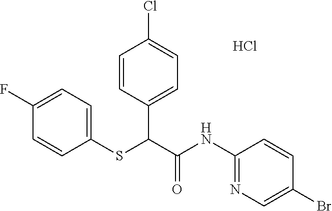

### Synthesis of N-(5-bromopyridin-2-yl)-2-(4-chlorophenyl)-2-(4-fluorophenylthio)acetamide hydrochloride

The title compound was generated from commercially available 2-bromo-2-(4-chlorophenyl)acetic acid, 4-fluorobenzenethiol and 5-bromopyridin-2-amine according to general procedure B described above. The intermediate 2-(4-chlorophenyl)-2-(4-fluorophenylthio)acetic acid was used in the subsequent synthetic transformation without further purification.

2-(4-Chlorophenyl)-2-(4-fluorophenylthio)acetic acid: The title compound was obtained from commercially available 2-bromo-2-(4-chlorophenyl)acetic acid (10.45 g, 41.89 mmol), 4-fluorobenzenethiol (5.50 g, 42.91 mmol) and sodium hydride (60% oil dispersion, 5.50 g, 137.50 mmol) according to the protocols as outlined in general procedure B above. This acid was used in the next synthetic step without further purification.

N-(5-Bromopyridin-2-yl)-2-(4-chlorophenyl)-2-(4-fluorophenylthio)acetamide hydrochloride: The title compound was obtained from 2-(4-chlorophenyl)-2-(4-fluorophenylthio)acetic acid (taken from the previous step, crude, 3.00 g, 9.65 mmol), 5-bromopyridin-2-amine (2.00 g, 11.56 mmol) and EDCI (3.50 g, 18.26 mmol) according to the protocols as outlined in general procedure B above. Spectroscopic Data: ¹H NMR (500 MHz, DMSO-d₆) δ ppm 5.56 (s, 1H), 7.14 (t, 2H), 7.35-7.44 (m, 4H), 7.51 (d, J=8.31 Hz, 2H), 7.97 (s, 2H), 8.40 (t, J=1.59 Hz, 1H), 10.66 (br. s., 1H), 11.20 (s, 1H).

### Example 15

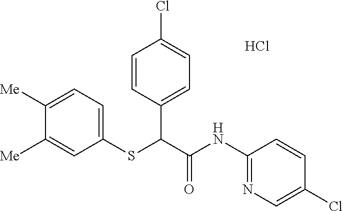

### Synthesis of 2-(4-chlorophenyl)-N-(5-chloropyridin-2-yl)-2-(3,4-dimethylphenylthio)acetamide hydrochloride

The title compound was generated from commercially available 2-bromo-2-(4-chlorophenyl)acetic acid, 3,4-dimethylbenzenethiol and 5-chloropyridin-2-amine according to general procedure B described above. The intermediate 2-(4-chlorophenyl)-2-(3,4-dimethylphenylthio)acetic acid was used in the subsequent synthetic transformation without further purification.

2-(4-Chlorophenyl)-2-(3,4-dimethylphenylthio)acetic acid: The title compound was obtained from commercially available 2-bromo-2-(4-chlorophenyl)acetic acid (11.47 g, 45.97 mmol), 3,4-dimethylbenzenethiol (6.50 g, 47.02 mmol) and sodium hydride (60% oil dispersion, 5.50 g, 137.50 mmol) according to the protocols as outlined in general procedure B above. This acid was used in the next synthetic step without further purification.

2-(4-Chlorophenyl)-N-(5-chloropyridin-2-yl)-2-(3,4-dimethylphenylthio)acetamide hydrochloride: The title compound was obtained from 2-(4-bromophenoxy)-2-(4-chlorophenyl)acetic acid (taken from the previous step, crude, 3.00 g, 9.78 mmol), 5-chloropyridin-2-amine (1.50 g, 11.67 mmol) and EDCI (3.50 g, 18.26 mmol) according to the protocols as outlined in general procedure B above. Spectroscopic Data: ¹H NMR (500 MHz, DMSO-d₆) δ ppm 2.11 (s, 3H), 2.11 (s, 3H), 5.55 (s, 1H), 6.97-7.04 (m, 1H), 7.04-7.09 (m, 1H), 7.15 (s, 1H), 7.32-7.44 (m, 2H), 7.46-7.61 (m, 2H), 7.86 (dd, J=8.93, 2.57 Hz, 1H), 8.02 (d, J=8.80 Hz, 1H), 8.34 (d, J=2.45 Hz, 1H), 8.70 (br. s., 2 H), 11.17 (s, 1H).

### Example 16

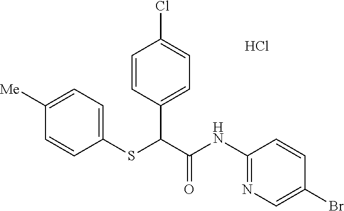

### Synthesis of N-(5-bromopyridin-2-yl)-2-(4-chlorophenyl)-2-(p-tolylthio)acetamide hydrochloride

The title compound was generated from commercially available 2-bromo-2-(4-chlorophenyl)acetic acid, 4-methylbenzenethiol and 5-bromopyridin-2-amine according to General procedure B described above. The intermediate 2-(4-chlorophenyl)-2-(p-tolylthio)acetic acid was used in the subsequent synthetic transformation without further purification.

2-(4-Chlorophenyl)-2-(p-tolylthio)acetic acid: The title compound was obtained from commercially available 2-bromo-2-(4-chlorophenyl)acetic acid (10.67 g, 42.77 mmol), 4-methylbenzenethiol (5.60 g, 45.09 mmol) and sodium hydride (60% oil dispersion, 5.50 g, 137.50 mmol) according to the protocols as outlined in general procedure B above. This acid was used in the next synthetic step without further purification.

2-(4-N-(5-Bromopyridin-2-yl)-2-(4-chlorophenyl)-2-(p-tolylthio)acetamide hydrochloride: The title compound was obtained from 2-(4-chlorophenyl)-2-(p-tolylthio)acetic acid (taken from the previous step, crude, 3.00 g, 10.25 mmol), 5-bromopyridin-2-amine (2.00 g, 11.56 mmol) and EDCI (3.50 g, 18.26 mmol) according to the protocols as outlined in general procedure B above. Spectroscopic Data: ¹H NMR (500 MHz, DMSO-d₆) δ ppm 2.22 (s, 3H), 5.51 (s, 1H), 7.10 (d, J=8.07 Hz, 2H), 7.24 (d, J=8.07 Hz, 2H), 7.35 (dd, J=16.87, 6.36 Hz, 1H), 7.38-7.44 (m, 2H), 7.51 (d, J=8.56 Hz, 2H), 7.90-8.01 (m, 1H), 8.42 (dd, J=2.08, 1.10 Hz, 1H), 11.15 (s, 1H).

### Example 17

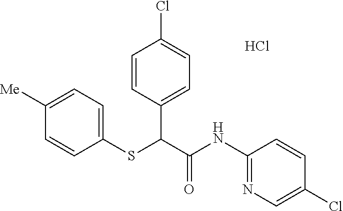

### Synthesis of 2-(4-chlorophenyl)-N-(5-chloropyridin-2-yl)-2-(p-tolylthio)acetamide hydrochloride

The title compound was generated from commercially available 2-bromo-2-(4-chlorophenyl)acetic acid, 4-methylbenzenethiol and 5-chloropyridin-2-amine according to general procedure B described above. The intermediate 2-(4-chlorophenyl)-2-(p-tolylthio)acetic acid was used in the subsequent synthetic transformation without further purification.

2-(4-Chlorophenyl)-2-(p-tolylthio)acetic acid: The title compound was obtained from commercially available 2-bromo-2-(4-chlorophenyl)acetic acid (10.67 g, 42.77 mmol), 4-methylbenzenethiol (5.60 g, 45.09 mmol) and sodium hydride (60% oil dispersion, 5.50 g, 137.50 mmol) according to the protocols as outlined in general procedure B above. This acid was used in the next synthetic step without further purification

2-(4-Chlorophenyl)-N-(5-chloropyridin-2-yl)-2-(p-tolylthio)acetamide hydrochloride: The title compound was obtained from 2-(4-chlorophenyl)-2-(p-tolylthio)acetic acid (taken from the previous step, crude, 3.00 g, 10.25 mmol), 5-chloropyridin-2-amine (1.50 g, 11.67 mmol) and EDCI (3.50 g, 18.26 mmol) according to the protocols as outlined in general procedure B above. Spectroscopic Data: ¹H NMR (500 MHz, DMSO-d₆) δ ppm 2.20 (s, 3H), 5.58 (s, 1H), 7.07 (d, J=7.82 Hz, 2H), 7.24 (d, J=8.07 Hz, 2H), 7.38 (d, J=8.56 Hz, 2H), 7.53 (d, J=8.56 Hz, 2H), 7.85 (dd, J=8.93, 2.57 Hz, 1H), 8.02 (d, J=9.05 Hz, 1H), 8.33 (d, J=2.69 Hz, 1H), 10.42 (br. s., 1H), 11.22 (s, 1H).

### Example 18

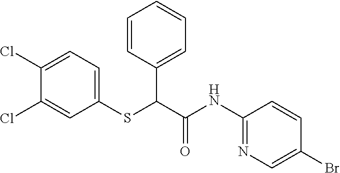

### Synthesis of N-(5-bromopyridin-2-yl)-2-(3,4-dichlorophenylthio)-2-phenylacetamide

The title compound was generated from commercially available 2-bromo-2-phenylacetic acid, 3,4-dichlorobenzenethiol, oxalyl chloride and 5-bromopyridin-2-amine according to general procedure A described above. The intermediate 2-(3,4-dichlorophenylthio)-2-phenylacetyl chloride was used in the subsequent synthetic transformation without further purification.

2-(3,4-Dichlorophenylthio)-2 phenylacetyl chloride: The title compound was obtained from commercially available 2-bromo-2-phenylacetic acid (12.00 g, 55.80 mmol), 3,4-dichlorobenzenethiol (10.00 g, 55.84 mmol), sodium hydride (60% oil dispersion, 5.00 g, 125.00 mmol) and oxalyl chloride (2 M in dichloromethane, 50.00 mL, 100.00 mmol) according to the protocols as outlined in general procedure A above. This acid chloride was used in the next synthetic step without further purification.

N-(5-Bromopyridin-2-yl)-2-(3,4-dichlorophenylthio)-2-phenylacetamide: The title compound was obtained from 2-(3,4-dichlorophenylthio)-2-phenylacetyl chloride (taken from the previous step, crude, 3.31 g, 10.00 mmol), 5-bromopyridin-2-amine (1.90 g, 10.98 mmol) and Hünig's base (4.00 mL, 22.93 mmol) according to the protocols as outlined in general procedure A above. Free base was obtained. Spectroscopic Data: ¹H NMR (500 MHz, DMSO-d₆) δ ppm 5.60 (s, 1H), 7.31 (t, J=7.21 Hz, 2H), 7.36 (t, J=7.34 Hz, 2H), 7.46-7.67 (m, 4H), 7.87-8.10 (m, 2H), 8.43 (s, 1H), 11.15 (s, 1H).

### Example 19

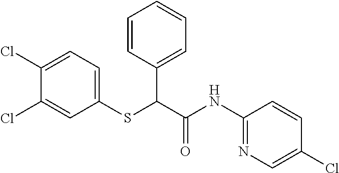

### Synthesis of N-(5-chloropyridin-2-yl)-2-(3,4-dichlorophenylthio)-2-phenylacetamide

The title compound was generated from commercially available 2-bromo-2-phenylacetic acid, 3,4-dichlorobenzenethiol, oxalyl chloride and 5-chloropyridin-2-amine according to general procedure A described above. The intermediate 2-(3,4-dichlorophenylthio)-2-phenylacetyl chloride was used in the subsequent synthetic transformation without further purification.

2-(3,4-Dichlorophenylthio)-2 phenylacetyl chloride: The title compound was obtained from commercially available 2-bromo-2-phenylacetic acid (12.00 g, 55.80 mmol), 3,4-dichlorobenzenethiol (10.00 g, 55.84 mmol), sodium hydride (60% oil dispersion, 5.00 g, 125.00 mmol) and oxalyl chloride (2 M in dichloromethane, 50.00 mL, 100.00 mmol) according to the protocols as outlined in general procedure A above. This acid chloride was used in the next synthetic step without further purification.

N-(5-Chloropyridin-2-yl)-2-(3,4-dichlorophenylthio)-2-phenylacetamide: The title compound was obtained from 2-(3,4-dichlorophenylthio)-2-phenylacetyl chloride (taken from the previous step, crude, 3.31 g, 10.00 mmol), 5-chloropyridin-2-amine (1.50 g, 11.67 mmol) and Hünig's base (4.00 mL, 22.93 mmol) according to the protocols as outlined in general procedure A above. Free base was obtained. Spectroscopic Data: ¹H NMR (500 MHz, DMSO-d₆) δ ppm 5.61 (s, 1H), 7.31 (t, J=6.97 Hz, 2H), 7.36 (t, J=7.34 Hz, 2H), 7.47-7.63 (m, 4H), 7.88 (dd, J=8.93, 2.32 Hz, 1H), 8.02 (d, J=9.05 Hz, 1H), 8.36 (d, J=2.20 Hz, 1H), 11.15 (s, 1H).

### Example 20

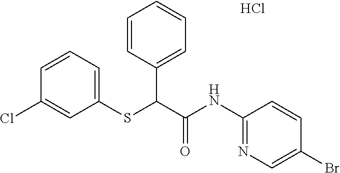

### Synthesis of N-(5-bromopyridin-2-yl)-2-(3-chlorophenylthio)-2-phenylacetamide hydrochloride

The title compound was generated from commercially available 2-bromo-2-phenylacetic acid, 3-chlorobenzenethiol, oxalyl chloride and 5-bromopyridin-2-amine according to general procedure A described above. The intermediate 2-(3-chlorophenylthio)-2-phenylacetyl chloride was used in the subsequent synthetic transformation without further purification.

2-(3-Chlorophenylthio)-2 phenylacetyl chloride: The title compound was obtained from commercially available 2-bromo-2-phenylacetic acid (12.00 g, 55.80 mmol), 3-chlorobenzenethiol (8.07 g, 55.80 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

N-(5-Bromopyridin-2-yl)-2-(3-chlorophenylthio)-2-phenylacetamide hydrochloride: The title compound was obtained from 2-(3-chlorophenylthio)-2-phenylacetyl chloride (taken from the previous step, crude, 2.80 g, 9.42 mmol), 5-bromopyridin-2-amine (1.90 g, 10.98 mmol) and Hünig's base (4.00 mL, 22.93 mmol) according to the protocols as outlined in general procedure A above. Spectroscopic Data: ¹H NMR (500 MHz, DMSO-d₆) δ ppm 5.71 (s, 1H), 7.20-7.32 (m, 3H), 7.35 (t, J=7.46 Hz, 2H), 7.39-7.43 (m, 1H), 7.55 (d, J=7.09 Hz, 1H), 7.92-8.01 (m, 2H), 8.42 (t, 1H), 8.97 (br. s., 1H), 11.22 (s, 1H).

### Example 21

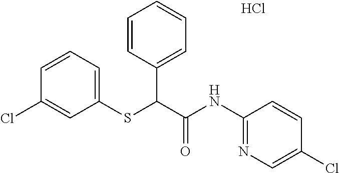

### Synthesis of 2-(3-chlorophenylthio)-N-(5-chloropyridin-2-yl)-2-phenylacetamide hydrochloride

The title compound was generated from commercially available 2-bromo-2-phenylacetic acid, 3-chlorobenzenethiol, oxalyl chloride and 5-chloropyridin-2-amine according to general procedure A described above. The intermediate 2-(3-chlorophenylthio)-2-phenylacetyl chloride was used in the subsequent synthetic transformation without further purification.

2-(3-Chlorophenylthio)-2 phenylacetyl chloride: The title compound was obtained from commercially available 2-bromo-2-phenylacetic acid (12.00 g, 55.80 mmol), 3-chlorobenzenethiol (8.07 g, 55.80 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

2-(3-Chlorophenylthio)-N-(5-chloropyridin-2-yl)-2-phenylacetamide hydrochloride: The title compound was obtained from 2-(3-chlorophenylthio)-2-phenylacetyl chloride (taken from the previous step, crude, 2.80 g, 9.42 mmol), 5-chloropyridin-2-amine (1.50 g, 11.67 mmol) and Hünig's base (4.00 mL, 22.93 mmol) according to the protocols as outlined in general procedure A above. Spectroscopic Data: ¹H NMR (500 MHz, DMSO-d₆) ¹H NMR (500 MHz, <dmso>) δ ppm 5.67 (s, 1H), 6.00 (br. s., 1H), 7.24-7.32 (m, 3H), 7.36 (t, J=7.34 Hz, 2H), 7.39-7.43 (m, 1H), 7.55 (d, J=7.09 Hz, 2H), 7.88 (dd, J=9.05, 2.69 Hz, 1H), 8.02 (d, J=9.05 Hz, 1H), 8.36 (d, J=2.69 Hz, 1H), 11.20 (s, 1H).

### Example 22

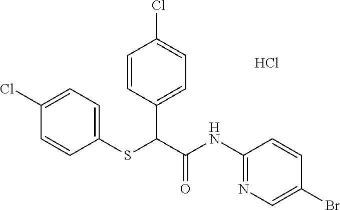

### Synthesis of N-(5-bromopyridin-2-yl)-2-(4-chlorophenyl)-2-(4-chlorophenylthio)acetamide hydrochloride

The title compound was generated from commercially available 2-bromo-2-(4-chlorophenyl)acetic acid, 4-chlorobenzenethiol, oxalyl chloride and 5-bromopyridin-2-amine according to general procedure A described above. The intermediate 2-(4-chlorophenyl)-2-(4-chlorophenylthio)acetyl chloride was used in the subsequent synthetic transformation without further purification.

2-(4-Chlorophenyl)-2-(4-chlorophenylthio)acetyl chloride: The title compound was obtained from commercially available 2-bromo-2-(4-chlorophenyl)acetic acid (12.00 g, 48.10 mmol), 4-chlorobenzenethiol (6.96 g, 48.13 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

N-(5-Bromopyridin-2-yl)-2-(4-chlorophenyl)-2-(4-chlorophenylthio)acetamide hydrochloride: The title compound was obtained from 2-(4-chlorophenyl)-2-(4-chlorophenylthio)acetyl chloride (taken from the previous step, crude, 3.30 g, 9.95 mmol), 5-bromopyridin-2-amine (1.90 g, 10.98 mmol) and Hünig's base (4.00 mL, 22.93 mmol) according to the protocols as outlined in general procedure A above. Spectroscopic Data: ¹H NMR (500 MHz, DMSO-d₆) δ ppm 5.63 (s, 1H), 6.47 (br. s., 1H), 7.35 (s, 4H), 7.39-7.42 (m, 1H), 7.53 (d, J=8.56 Hz, 2H), 7.93-7.97 (m, 1H), 7.97-8.01 (m, 1H), 8.42 (d, J=3.18 Hz, 1H), 11.22 (s, 1H).

### Example 23

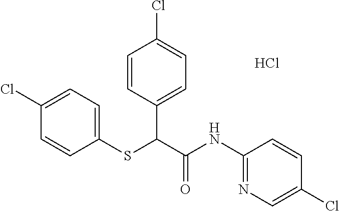

### Synthesis of 2-(4-chlorophenyl)-2-(4-chlorophenylthio)-N-(5-chloropyridin-2-yl)acetamide hydrochloride

The title compound was generated from commercially available 2-bromo-2-(4-chlorophenyl)acetic acid, 4-chlorobenzenethiol, oxalyl chloride and 5-chloropyridin-2-amine according to general procedure A described above. The intermediate 2-(4-chlorophenyl)-2-(4-chlorophenylthio)acetyl chloride was used in the subsequent synthetic transformation without further purification.

2-(4-Chlorophenyl)-2-(4-chlorophenylthio)acetyl chloride: The title compound was obtained from commercially available 2-bromo-2-(4-chlorophenyl)acetic acid (12.00 g, 48.10 mmol), 4-chlorobenzenethiol (6.96 g, 48.13 mmol), sodium hydride (60% oil dispersion, 5.50 g, 137.50 mmol) and oxalyl chloride (2 M in dichloromethane, 50.00 mL, 100.00 mmol) according to the protocols as outlined in general procedure A above. This acid chloride was used in the next synthetic step without further purification.

2-(4-Chlorophenyl)-2-(4-chlorophenylthio)-N-(5-chloropyridin-2-yl)acetamide hydrochloride: The title compound was obtained from 2-(4-chlorophenyl)-2-(4-chlorophenylthio)acetyl chloride (taken from the previous step, crude, 3.30 g, 9.95 mmol), 5-chloropyridin-2-amine (1.50 g, 11.67 mmol) and Hünig's base (4.00 mL, 22.93 mmol) according to the protocols as outlined in general procedure A above. Spectroscopic Data: ¹H NMR (500 MHz, DMSO-d₆) δ ppm 5.61 (s, 1H), 6.94 (br. s., 1H), 7.33-7.38 (m, 4 H), 7.41 (d, J=8.31 Hz, 2H), 7.53 (d, J=8.56 Hz, 2H), 7.88 (dd, J=8.93, 2.57 Hz, 1H), 8.01 (d, J=8.80 Hz, 1H), 8.35 (d, J=2.45 Hz, 1H), 11.22 (s, 1H).

### Example 24

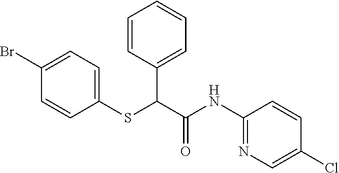

### Synthesis of 2-(4-bromophenylthio)-N-(5-chloropyridin-2-yl)-2-phenylacetamide

The title compound was generated from commercially available 2-bromo-2-phenylacetic acid, 4-bromobenzenethiol, oxalyl chloride and 5-chloropyridin-2-amine according to general procedure A described above. The intermediate 2-(4-bromophenylthio)-2-phenylacetyl chloride was used in the subsequent synthetic transformation without further purification.

2-(4-Bromophenylthio)-2 phenylacetyl chloride: The title compound was obtained from commercially available 2-bromo-2-phenylacetic acid (10.00 g, 46.50 mmol), 4-bromobenzenethiol (8.80 g, 46.56 mmol), sodium hydride (60% oil dispersion, 5.50 g, 137.50 mmol) and oxalyl chloride (2 M in dichloromethane, 60.00 mL, 120.00 mmol) according to the protocols as outlined in general procedure A above. This acid chloride was used in the next synthetic step without further purification.

2-(4-Bromophenylthio)-N-(5-chloropyridin-2-yl)-2-phenylacetamide: The title compound was obtained from 2-(4-bromophenylthio)-2-phenylacetyl chloride (taken from the previous step, crude, 3.40 g, 9.95 mmol), 5-chloropyridin-2-amine (1.28 g, 9.96 mmol) and Hünig's base (5.00 mL, 28.66 mmol) according to the protocols as outlined in general procedure A above. Free base was obtained. Spectroscopic Data: ¹H NMR (500 MHz, DMSO-d₆) δ ppm 5.58 (s, 1H), 7.31 (d, J=8.56 Hz, 3H), 7.38 (t, J=7.34 Hz, 2H), 7.52 (d, J=8.56 Hz, 2H), 7.56 (d, J=7.34 Hz, 2H), 7.90 (dd, J=8.93, 2.57 Hz, 1H), 8.06 (d, J=8.80 Hz, 1H), 8.38 (d, J=2.45 Hz, 1H), 11.19 (s, 1H).

### Example 25

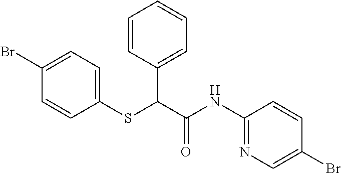

### Synthesis of 2-(4-bromophenylthio)-N-(5-bromopyridin-2-yl)-2-phenylacetamide

The title compound was generated from commercially available 2-bromo-2-phenylacetic acid, 4-bromobenzenethiol, oxalyl chloride and 5-bromopyridin-2-amine according to general procedure A described above. The intermediate 2-(4-bromophenylthio)-2-phenylacetyl chloride was used in the subsequent synthetic transformation without further purification.

2-(4-Bromophenylthio)-2 phenylacetyl chloride: The title compound was obtained from commercially available 2-bromo-2-phenylacetic acid (10.00 g, 46.50 mmol), 4-bromobenzenethiol (8.80 g, 46.56 mmol), sodium hydride (60% oil dispersion, 5.50 g, 137.50 mmol) and oxalyl chloride (2 M in dichloromethane, 60.00 mL, 120.00 mmol) according to the protocols as outlined in general procedure A above. This acid chloride was used in the next synthetic step without further purification.

2-(4-Bromophenylthio)-N-(5-bromopyridin-2-yl)-2-phenylacetamide: The title compound was obtained from 2-(4-bromophenylthio)-2-phenylacetyl chloride (taken from the previous step, crude, 3.40 g, 9.95 mmol), 5-bromopyridin-2-amine (1.73 g, 10.00 mmol) and Hünig's base (5.00 mL, 28.66 mmol) according to the protocols as outlined in general procedure A above. Free base was obtained. Spectroscopic Data: ¹H NMR (500 MHz, DMSO-d₆) δ ppm 5.57 (s, 1H), 7.24-7.34 (m, 2H), 7.37 (t, J=7.34 Hz, 2H), 7.53 (dd, J=13.94, 7.83 Hz, 4H), 8.00 (s, 2H), 8.45 (s, 1H), 11.18 (s, 1H).

### Example 26

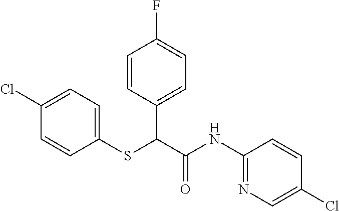

### Synthesis of 2-(4-chlorophenylthio)-N-(5-chloropyridin-2-yl)-2-(4-fluorophenyl)acetamide

The title compound was generated from commercially available 2-bromo-2-(4-fluorophenyl)acetic acid, 4-chlorobenzenethiol, oxalyl chloride and 5-chloropyridin-2-amine according to general procedure A described above. The intermediate 2-(4-chlorophenylthio)-2-(4-fluorophenyl)acetyl chloride was used in the subsequent synthetic transformation without further purification.

2-(4-Chlorophenylthio)-2-(4-fluorophenyl)acetyl chloride: The title compound was obtained from commercially available 2-bromo-2-phenylacetic acid (12.80 g, 54.94 mmol), 4-chlorobenzenethiol (8.00 g, 55.32 mmol), sodium hydride (60% oil dispersion, 5.30 g, 132.50 mmol) and oxalyl chloride (2 M in dichloromethane, 60.00 mL, 120.00 mmol) according to the protocols as outlined in general procedure A above. This acid chloride was used in the next synthetic step without further purification.

2-(4-Chlorophenylthio)-N-(5-chloropyridin-2-yl)-2-(4-fluorophenyl)acetamide: The title compound was obtained from 2-(4-chlorophenylthio)-2-(4-fluorophenyl)acetyl chloride (taken from the previous step, crude, 3.15 g, 10.00 mmol), 5-chloropyridin-2-amine (1.28 g, 10.00 mmol) and Hünig's base (5.00 mL, 28.66 mmol) according to the protocols as outlined in general procedure A above. Free base was obtained. Spectroscopic Data: ¹H NMR (500 MHz, DMSO-d₆) δ ppm 5.56 (s, 1H), 7.21 (t, J=8.80 Hz, 2H), 7.38 (dd, J=19.07, 2.45 Hz, 4H), 7.57 (dd, J=8.44, 5.50 Hz, 2H), 7.90 (dd, J=9.05, 2.45 Hz, 1H), 8.06 (d, J=8.80 Hz, 1H), 8.38 (d, J=2.20 Hz, 1H), 11.17 (s, 1H).

### Example 27

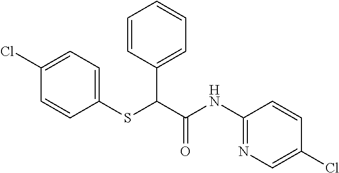

### Synthesis of 2-(4-chlorophenylthio)-N-(5-chloropyridin-2-yl)-2-phenylacetamide

The title compound was generated from commercially available 2-bromo-2-phenylacetic acid, 4-chlorobenzenethiol, oxalyl chloride and 5-chloropyridin-2-amine according to general procedure A described above. The intermediate 2-(4-chlorophenylthio)-2-phenylacetyl chloride was used in the subsequent synthetic transformation without further purification.

2-(4-Chlorophenylthio)-2 phenylacetyl chloride: The title compound was obtained from commercially available 2-bromo-2-phenylacetic acid (12.00 g, 55.81 mmol), 4-chlorobenzenethiol (8.00 g, 55.32 mmol), sodium hydride (60% oil dispersion, 5.00 g, 125.00 mmol) and oxalyl chloride (2 M in dichloromethane, 60.00 mL, 120.00 mmol) according to the protocols as outlined in general procedure A above. This acid chloride was used in the next synthetic step without further purification.

2-(4-Chlorophenylthio)-N-(5-chloropyridin-2-yl)-2-phenylacetamide: The title compound was obtained from 2-(4-chlorophenylthio)-2-phenylacetyl chloride (taken from the previous step, crude, 3.00 g, 10.09 mmol), 5-chloropyridin-2-amine (1.28 g, 9.96 mmol) and Hünig's base (5.00 mL, 28.66 mmol) according to the protocols as outlined in general procedure A above. Free base was obtained. Spectroscopic Data: ¹H NMR (500 MHz, DMSO-d₆) δ ppm 5.57 (br. s., 1H), 7.26-7.46 (m, 7H), 7.55 (d, J=7.34 Hz, 2H), 7.90 (d, J=1.22 Hz, 1H), 8.06 (d, J=8.80 Hz, 1H), 8.37 (br. s., 1H), 11.16 (br. s., 1H).

### Example 28

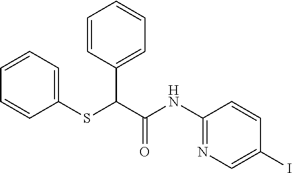

### Synthesis of N-(5-iodopyridin-2-yl)-2-phenyl-2-(phenylthio)acetamide

The title compound was generated from commercially available 2-phenyl-2-(phenylthio)acetic acid, oxalyl chloride and 5-iodopyridin-2-amine according to general procedure A described above. The intermediate 2-phenyl-2-(phenylthio)acetyl chloride was used in the subsequent synthetic transformation without further purification.

2-Phenyl-2-(phenylthio)acetyl chloride: The title compound was obtained from commercially available 2-phenyl-2-(phenylthio)acetic acid (25.00 g, 102.33 mmol) and oxalyl chloride (2 M in dichloromethane, 100.00 mL, 200.00 mmol) according to the protocols as outlined in general procedure A above. This acid chloride was used in the next synthetic step without further purification.

N-(5-Iodopyridin-2-yl)-2-phenyl-2-(phenylthio)acetamide: The title compound was obtained from 2-phenyl-2-(phenylthio)acetic acid (taken from the previous step, crude, 2.62 g, 10.72 mmol), 5-iodopyridin-2-amine (2.00 g, 9.09 mmol) and Hünig's base (4.00 mL, 22.93 mmol) according to the protocols as outlined in general procedure A above. Free base was obtained. Spectroscopic Data: ¹H NMR (500 MHz, DMSO-d₆) δ ppm 5.61 (s, 1H), 7.23 (d, J=7.34 Hz, 1H), 7.30 (t, J=7.70 Hz, 3H), 7.33-7.40 (m, 4H), 7.56 (d, J=7.09 Hz, 2H), 7.89 (d, J=8.56 Hz, 1H), 8.10 (dd, J=8.80, 2.20 Hz, 1H), 8.53 (d, J=2.44 Hz, 1H), 11.13 (s, 1H).

### General procedure for the Synthesis of 2-(Substituted Phenyl)-3-(substituted phenyl)-N-(substituted pyridin-2-yl)acetamide Hydrochloride

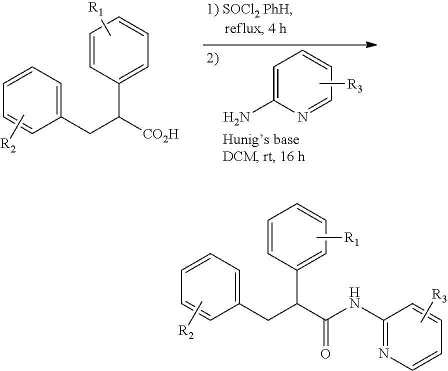

An appropriately substituted carboxylic acid was converted into the corresponding acid chloride by the method published in Newcomb et al. The acid chloride (1 eq) and an appropriately substituted 2-aminopyridine (1.5-2.0 eq) were dissolved in 50 ml of dichloromethane at room temperature. Hünig's base (3.50 mL) was added, and the resulting reaction mixture was stirred at room temperature for 14 hours. The reaction mixture was then concentrated and diluted with ethyl acetate. The organic phase was washed sequentially with water and brine, then dried with sodium sulfate and concentrated. Flash chromatography (30% EtOAc/hexanes) gave the desired free base, which was mixed with HCl in MeOH. Concentration and recrystallization from ether gave the final desired product in HCl salt form.

### Example 29

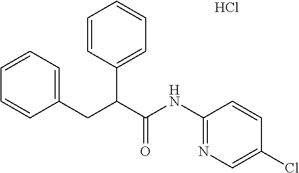

### Synthesis of N-(5-chloropyridin-2-yl)-2,3-diphenylpropanamide hydrochloride

The title compound was generated from commercially available 2,3-diphenylpropanoic acid, thionyl chloride and 5-chlororopyridin-2-amine according to the general procedure described above. The intermediate 2,3-diphenylpropanoic acid chloride was used in the subsequent synthetic transformation without further purification.

2,3-Diphenylpropanoic acid chloride: The title compound was obtained from commercially available 2,3-diphenylpropanoic acid (25.00 g, 110.49 mmol) and thionyl chloride (20.00 mL, 274.19 mmol) according to the protocols as outlined in the general procedure above. This acid chloride was used in the next synthetic step without further purification.

N-(5-Chloropyridin-2-yl)-2,3-diphenylpropanamide hydrochloride: The title compound was obtained from 2,3-diphenylpropanoic acid chloride (taken from the previous step, crude, 2.50 g, 10.22 mmol), 5-chloropyridin-2-amine (1.28 g, 10.00 mmol) and Hünig's base (3.50 mL, 20.09 mmol) according to the protocols as outlined in the general procedure above. Spectroscopic Data: ¹H NMR (500 MHz, DMSO-d₆) δ ppm 2.95 (dd, J=13.69, 6.11 Hz, 1H), 3.40 (dd, J=13.57, 9.17 Hz, 1H), 4.26 (dd, J=9.17, 6.24 Hz, 1H), 7.16-7.25 (m, 5H), 7.30 (t, J=7.58 Hz, 3H), 7.43 (d, J=7.09 Hz, 2H), 7.83 (dd, J=9.05, 2.69 Hz, 1H), 8.06 (d, J=9.05 Hz, 1H), 8.29 (d, J=2.20 Hz, 1H), 10.82 (s, 1H).

### Example 30

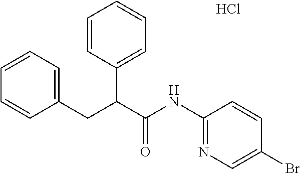

### Synthesis of N-(5-bromopyridin-2-yl)-2,3-diphenylpropanamide hydrochloride

The title compound was generated from commercially available 2,3-diphenylpropanoic acid, thionyl chloride and 5-bromoropyridin-2-amine according to the general procedure described above. The intermediate 2,3-diphenylpropanoic acid chloride was used in the subsequent synthetic transformation without further purification.

2,3-Diphenylpropanoic acid chloride: The title compound was obtained from commercially available 2,3-diphenylpropanoic acid (25.00 g, 110.49 mmol) and thionyl chloride (20.00 mL, 274.19 mmol) according to the protocols as outlined in the general procedure above. This acid chloride was used in the next synthetic step without further purification.

N-(5-Bromopyridin-2-yl)-2,3-diphenylpropanamide hydrochloride: The title compound was obtained from 2,3-diphenylpropanoic acid chloride (taken from the previous step, crude, 2.50 g, 10.22 mmol), 5-bromopyridin-2-amine (1.63 g, 9.42 mmol) and Hünig's base (3.50 mL, 20.09 mmol) according to the protocols as outlined in the general procedure above. Spectroscopic Data: ¹H NMR (500 MHz, DMSO-d₆) δ ppm 2.95 (dd, J=13.69, 6.11 Hz, 1H), 3.40 (dd, J=13.69, 9.29 Hz, 1H), 4.26 (dd, J=9.17, 6.24 Hz, 1H), 7.17-7.24 (m, 5H), 7.30 (t, J=7.58 Hz, 3H), 7.43 (d, J=7.09 Hz, 2H), 7.91-7.96 (m, 1H), 7.98-8.06 (m, 1H), 8.36 (d, J=2.45 Hz, 1H), 10.82 (s, 1H).

### Example 31

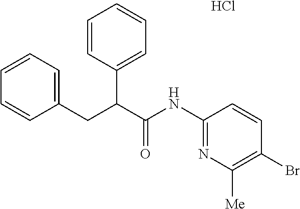

### Synthesis of N-(5-bromo-6-methylpyridin-2-yl)-2,3-diphenylpropanamide hydrochloride

The title compound was generated from commercially available 2,3-diphenylpropanoic acid, thionyl chloride and 5-bromo-6-methylpyridin-2-amine according to the general procedure described above. The intermediate 2,3-diphenylpropanoic acid chloride was used in the subsequent synthetic transformation without further purification.

2,3-Diphenylpropanoic acid chloride: The title compound was obtained from commercially available 2,3-diphenylpropanoic acid (25.00 g, 110.49 mmol) and thionyl chloride (20.00 mL, 274.19 mmol) according to the protocols as outlined in the general procedure above. This acid chloride was used in the next synthetic step without further purification.

N-(5-Bromo-6-methylpyridin-2-yl)-2,3-diphenylpropanamide hydrochloride: The title compound was obtained from 2,3-diphenylpropanoic acid chloride (taken from the previous step, crude, 2.50 g, 10.22 mmol), 5-bromo-6-methylpyridin-2-amine (1.87 g, 10.00 mmol) and Hünig's base (3.00 mL, 17.22 mmol) according to the protocols as outlined in the general procedure above. Spectroscopic Data: ¹H NMR (500 MHz, DMSO-d₆) δ ppm 2.43 (s, 3H), 2.93 (dd, J=13.69, 6.11 Hz, 1H), 3.39 (dd, J=13.69, 9.54 Hz, 1H), 4.27 (dd, 1H), 6.61 (br. s., 1H), 7.08-7.14 (m, 1H), 7.16-7.24 (m, 5H), 7.29 (t, J=7.58 Hz, 2H), 7.43 (d, J=7.34 Hz, 2H), 7.78-7.84 (m, 1H), 7.85-7.90 (m, 1H), 10.73 (s, 1H).

### Example 32

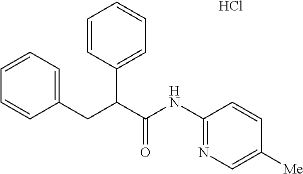

### Synthesis of N-(5-methylpyridin-2-yl)-2,3-diphenylpropanamide hydrochloride

The title compound was generated from commercially available 2,3-diphenylpropanoic acid, thionyl chloride and 5-methylpyridin-2-amine according to the general procedure described above. The intermediate 2,3-diphenylpropanoic acid chloride was used in the subsequent synthetic transformation without further purification.

2,3-Diphenylpropanoic acid chloride: The title compound was obtained from commercially available 2,3-diphenylpropanoic acid (25.00 g, 110.49 mmol) and thionyl chloride (20.00 mL, 274.19 mmol) according to the protocols as outlined in the general procedure above. This acid chloride was used in the next synthetic step without further purification.

N-(5-Methylpyridin-2-yl)-2,3-diphenylpropanamide hydrochloride: The title compound was obtained from 2,3-diphenylpropanoic acid chloride (taken from the previous step, crude, 2.50 g, 10.22 mmol), 5-methylpyridin-2-amine (1.08 g, 10.00 mmol) and Hünig's base (3.50 mL, 20.09 mmol) according to the protocols as outlined in the general procedure above. Spectroscopic Data: ¹H NMR (500 MHz, DMSO-d₆) δ ppm 2.26 (s, 3H), 3.04 (dd, J=13.82, 6.72 Hz, 1H), 3.45 (dd, J=13.82, 8.68 Hz, 1H), 4.46 (t, J=7.70 Hz, 1H), 6.28 (br s, 1H), 7.10-7.15 (m, 1H), 7.17-7.26 (m, 5H), 7.27-7.34 (m, 2H), 7.50 (d, J=7.34 Hz, 2H), 7.84 (d, J=8.80 Hz, 1H), 8.00 (d, J=8.56 Hz, 1H), 8.15 (s, 1H), 12.51 (br. s., 1H).

### Example 33

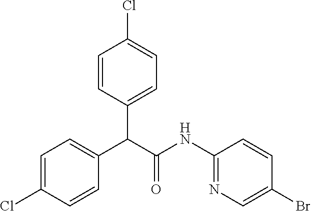

### Synthesis of N-(5-bromopyridin-2-yl)-2,2-bis(4-chlorophenyl)acetamide

The title compound was generated from commercially available 2,3-diphenylpropanoic acid, thionyl chloride and 5-cyanopyridin-2-amine according to the general procedure described above. The intermediate 2,3-diphenylpropanoic acid chloride was used in the subsequent synthetic transformation without further purification.

2,2-bis(4-Chlorophenyl)acetic acid chloride: The title compound was obtained from commercially available 2,2-bis(4-chlorophenyl)acetic acid (10.00 g, 35.57 mmol) and thionyl chloride (20.00 mL, 274.19 mmol) according to the protocols as outlined in the general procedure above. This acid chloride was used in the next synthetic step without further purification.

N-(5-Bromopyridin-2-yl)-2,2-bis(4-chlorophenyl)acetamide: The title compound was obtained from 2,2-bis(4-chlorophenyl)acetic acid chloride (taken from the previous step, crude, 3.00 g, 10.01 mmol), 5-bromopyridin-2-amine (1.90 g, 10.98 mmol) and Hünig's base (3.00 mL, 17.22 mmol) according to the protocols as outlined in the general procedure above. Spectroscopic Data: ¹H NMR (500 MHz, DMSO-d₆) δ ppm 5.39 (s, 1 H), 7.27-7.36 (m, 4H), 7.37-7.46 (m, 4H), 7.95-8.03 (m, 1H), 8.04-8.11 (m, 1H), 8.43 (d, J=2.45 Hz, 1H), 11.17 (s, 1H).

### Example 34

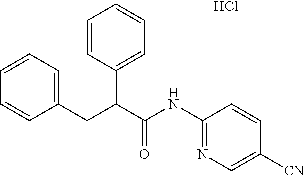

### Synthesis of N-(5-cyanopyridin-2-yl)-2,3-diphenylpropanamide hydrochloride

The title compound was generated from commercially available 2,3-diphenylpropanoic acid, thionyl chloride and 5-cyanopyridin-2-amine according to the general procedure described above. The intermediate 2,3-diphenylpropanoic acid chloride was used in the subsequent synthetic transformation without further purification.

2,3-Diphenylpropanoic acid chloride: The title compound was obtained from commercially available 2,3-diphenylpropanoic acid (25.00 g, 110.49 mmol) and thionyl chloride (20.00 mL, 274.19 mmol) according to the protocols as outlined in the general procedure above. This acid chloride was used in the next synthetic step without further purification.

N-(5-Cyanopyridin-2-yl)-2,3-diphenylpropanamide hydrochloride: The title compound was obtained from 2,3-diphenylpropanoic acid chloride (taken from the previous step, crude, 3.00 g, 12.26 mmol), 5-cyanopyridin-2-amine (2.00 g, 16.79 mmol) and Hünig's base (3.50 mL, 20.09 mmol) according to the protocols as outlined in the general procedure above. Spectroscopic Data: ¹H NMR (500 MHz, DMSO-d₆) δ ppm 2.96 (dd, J=13.69, 6.11 Hz, 1H), 3.41 (dd, J=13.69, 9.29 Hz, 1H), 4.29 (dd, J=9.05, 6.36 Hz, 1H), 5.58-5.75 (m, 1H), 7.07-7.15 (m, 1H), 7.16-7.26 (m, 5H), 7.31 (t, J=7.58 Hz, 3 H), 7.44 (d, J=7.34 Hz, 2H), 8.07 (d, J=8.80 Hz, 1H), 8.17 (dd, J=8.80, 2.45 Hz, 1H), 8.73 (d, J=1.96 Hz, 1H), 10.94 (s, 1H).

Unless otherwise indicated, the following terms as used throughout this specification have the following meanings:

DCM refers to dichloromethane,

DMSO refers to dimethyl sulfoxide,

EDCI refers to 1-ethyl-3-(3-dimethylaminopropyl)carbodiimide,

THF refers to tetrahydrofuran,

EtOAc refers to ethylacetate,

“Me” refers to methyl.,

“Ph” refers to phenyl.,

“Pharmaceutically acceptable salt” refers to those salts which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

“Alkyl” refers to a straight-chain, branched or cyclic saturated aliphatic hydrocarbon. Preferably, the alkyl group has 1 to 12 carbons. More preferably, it is a lower alkyl of from 1 to 7 carbons, most preferably 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents selected from the group consisting of hydroxyl, cyano, alkoxy, ═O, ═S, NO₂, halogen, dimethyl amino and SH.

“Alkenyl” refers to a straight-chain, branched or cyclic unsaturated hydrocarbon group containing at least one carbon—carbon double bond. Preferably, the alkenyl group has 2 to 12 carbons. More preferably it is a lower alkenyl of from 2 to 7 carbons, most preferably 2 to 4 carbons. The alkenyl group may be optionally substituted with one or more substituents selected from the group consisting of hydroxyl, cyano, alkoxy, O, S, NO₂, halogen, dimethyl amino and SH.

“Alkynyl” refers to a straight-chain, branched or cyclic unsaturated hydrocarbon containing at least one carbon—carbon triple bond. Preferably, the alkynyl group has 2 to 12 carbons. More preferably it is a lower alkynyl of from 2 to 7 carbons, most preferably 2 to 4 carbons. The alkynyl group may be optionally substituted with one or more substituents selected from the group consisting of hydroxyl, cyano, alkoxy, O, S, NO₂, halogen, dimethyl amino and SH.

“Alkoxy” refers to an “O-alkyl” group.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SH, OH, NO₂, amine, thioether, cyano, alkoxy, alkyl, and amino.

“Alkaryl” refers to an alkyl that is covalently joined to an aryl group. Preferably, the alkyl is a lower alkyl.

“Aryloxy” refers to an “O-aryl” group.

“Arylalkyloxy” refers to an “O-alkaryl” group.

“Carbocyclic” refers to cyclic saturated or unsaturated aliphatic hydrocarbon and aryl hydrocarbon groups wherein the ring atoms are exclusively carbons, and comprises from 6 to 20 carbon atoms, including said ring atoms.

“Carbocyclic aryl” refers to an aryl group wherein the ring atoms are carbon.

“Heterocyclic” refers to cyclic groups wherein the ring atoms comprise carbon atoms and at least one oxygen, nitrogen, and/or sulfur atom and may be saturated, unsaturated, i.e. have one or more double bonds, or aryl, and comprises up to 20 carbon atoms and from 1 to 5 of the above heteroatoms.

“Heterocyclic aryl” refers to an aryl group having from 1 to 3 heteroatoms as ring atoms, the remainder of the ring atoms being carbon. Heteroatoms include oxygen, sulfur, and nitrogen.

“Hydrocarbyl” refers to a hydrocarbon radical having only carbon and hydrogen atoms. Preferably, the hydrocarbyl radical has from 1 to 20 carbon atoms, more preferably from 1 to 12 carbon atoms and most preferably from 1 to 7 carbon atoms.

“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halogen, nitrogen, oxygen, sulfur or phosphorus atom, e.g. fluoro, chloro, cyano, nitro, hydroxyl, phosphate, thiol, etc.

“Amide” refers to —C(O)—NH—R′, wherein R′ is alkyl, aryl, alkylaryl or hydrogen.

“Ester” refers to —C(O)—O—R′, wherein R′ is alkyl, aryl or alkylaryl.

“Thioamide” refers to —C(S)—NH—R′, wherein R′ is alkyl, aryl, alkylaryl or hydrogen.

“Thiol ester” refers to —C(O)—S—R′, wherein R′ is alkyl, aryl, alkylaryl or hydrogen.

“Amine” refers to a—N(R″)R′″ group, wherein R″ and R′ are independently selected from the group consisting of alkyl, aryl, and alkylaryl.

“Thioether” refers to —S—R″, wherein R″ is alkyl, aryl, or alkylaryl.

“Sulfonyl” refers to —S(O)₂—R″, where R″ is aryl, C(CN)═C-aryl, CH₂ CN, alkyaryl, sulfonamide, NH-alkyl, NH-alkylaryl, or NH-aryl.

Also, alternatively the substituent on the phenyl moiety may be referred to as an o, m or p substituent or a 2, 3 or 4 substituent, respectively. (Obviously, the 5 substituent is also a m substituent and the 6 substituent is an o substituent.)

The above compounds are evaluated for S1P3 activity according to the above assay: The results are reported in TABLE 2, below.

TABLE 2

| | FLIPR | | |
|---|---|---|---|
| Example | EC50 (nM)/(% Antagonism) | | |
| No. | S1P₁ | S1P₂ | S1P₃ |
| 1 | >8300 | >8300 | 128 |
| | | | (99) |
| 2 | >8300 | >8300 | 185 |
| | | | (100) |
| 3 | >8300 | >8300 | 166 |
| | | | (98) |
| 4 | >8300 | >8300 | 325 |
| | | | (97) |
| 5 | >8300 | >8300 | 90 |
| | | | (97) |
| 6 | >8300 | >8300 | 103 |
| | | | (97) |
| 7 | >8300 | >8300 | 316 |
| | | | (97) |
| 8 | >8300 | >8300 | 15 |
| | | | (97) |
| 9 | >8300 | >8300 | 27 |
| | | | (97) |
| 10 | >8300 | >8300 | 256 |
| | | | (100) |
| 11 | >8300 | >8300 | 361 |
| | | | (100) |
| 12 | >8300 | >8300 | 376 |
| | | | (99) |
| 13 | >8300 | >8300 | 198 |
| | | | (99) |
| 14 | >8300 | >8300 | 127 |
| | | | (99) |
| 15 | >8300 | >8300 | 240 |
| | | | (98) |
| 16 | >8300 | >8300 | 256 |
| | | | (100) |
| 17 | >8300 | >8300 | 322 |
| | | | (99) |
| 18 | >8300 | >8300 | 133 |
| | | | (97) |
| 19 | >8300 | >8300 | 106 |
| | | | (97) |
| 20 | >8300 | >8300 | 166 |
| | | | (97) |
| 21 | >8300 | >8300 | 208 |
| | | | (97) |
| 22 | >8300 | >8300 | 104 |
| | | | (97) |
| 23 | >8300 | >8300 | 173 |
| | | | (97) |
| 24 | >8300 | >8300 | 230 |
| | | | (99) |
| 25 | >8300 | >8300 | 286 |
| | | | (99) |
| 26 | >8300 | >8300 | 166 |
| | | | (102) |
| 27 | >8300 | >8300 | 160 |
| | | | (101) |
| 28 | >8300 | >8300 | 310 |
| | | | (101) |
| 29 | >8300 | >8300 | 198 |
| | | | (101) |
| 30 | >8300 | >8300 | 162 |
| | | | (100) |
| 31 | >8300 | >8300 | 201 |
| | | | (100) |
| 32 | >8300 | >8300 | 725 |
| | | | (99) |
| 33 | >8300 | >8300 | 1720 |
| | | | (94) |
| 34 | >8300 | >8300 | 1502 |
| | | | (52) |

As a result of the above activity of the compounds utilized in the method of the present invention, it is clear that such compounds may be used in treating and/or preventing the following diseases and conditions of the eye as well as other diseases and conditions discussed below. (It should be noted that “treating” means ameliorating and/or modulating a disease or disorder that exists in a subject (whether the subject is aware of the disease or disorder or not) or delaying the onset of the disease or disorder and “preventing” means preventing the recurrence, onset or development of one or more symptoms of a disease or disorder in a subject by administering one or more compounds of the invention.)

Glaucoma

S1P3 subtypes are expressed in primary human trabecular meshwork cells and S1P decreases outflow facility >30% in perfused porcine eyes (See IOVS 45, 2263; 2004) by altering paracellular permeability.

Dry Eye/Immunology

Induces lymphocyte sequestration without affecting T cell proliferation.

Angiogenesis Disorders

S1P3 receptor subtype is expressed in vascular endothelial cells and siRNA knockdown of S1P1 and S1P3 inhibits angiogenesis. S1P also promotes vascular endothelial cell migration and promotes barrier assembly and integrity.

Cardiovascular (S1P3)

S1P3 “knock out” mice lack S1P induced pulmonary edema.

The foregoing description details specific methods and compositions that can be employed to practice the present invention, and represents the best mode contemplated. Thus, however detailed the foregoing may appear in text, it should not be construed as limiting the overall scope hereof; rather, the ambit of the present invention was to be governed only by the lawful construction of the appended claims. In particular, the present invention includes, as novel compounds, having subtype-selective modulating activity of sphingosine-1-phosphate-3 (SIP₃) receptors, compounds selected from the group consisting of 2-(substituted)(arylmethyl, aryloxy, and arylthio)-N-(substituted pyridin-2-yl)-2-(substituted aryl) compounds, wherein said aryl is a carbocyclic aryl or a heterocyclic aryl, which is substituted with one or more radicals selected from the group consisting of alkyl, alkenyl, alkynyl, alkaryl, alkyloxy, aryloxy, arylalkyloxy, amine, amide, hydroxyl, halogen, nitrile, nitro, trifluoromethyl, carboxy, ester, thiolester, thioether and sulfonyl.

What is claimed is:

1. Compounds represented by the formula I having sphingosine-1-phosphate receptor antagonist biological activity or a pharmaceutically acceptable salt thereof: 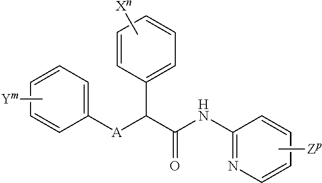
wherein:
A is O, S or (CR₂)ₐ wherein R is selected from the group consisting of H, or lower alkyl;
m, n, p are 0 or an integer of from 1 to 5;
a is 0 or 1;
wherein when a is 1;
X and Y are independently selected from the group consisting of alkyl, alkyloxy, hydroxyl, halogen, nitrile and trifluoromethyl;
Z is selected from the group consisting of alkyl, alkyloxy, hydroxyl, halogen, nitrile and trifluoromethyl and; and
when a is 0;
X, Y are independently selected from the group consisting of alkyl, alkyloxy, hydroxyl, halogen, nitrile and trifluoromethyl;
Z is selected from the group consisting of alkyl, alkyloxy, hydroxyl, halogen, nitrile and trifluoromethyl;
provided however that when a is 0 and when the pyridyl ring is (a) 5-chloro pyridyl, then both (i) m and n are not 0 and (ii) X and Y are not chloro or methyl or (b) 5-bromo or iodo pyridyl, than both m and n are not 0.

2. (canceled)

3. The compound of claim 1 wherein X and Y are independently selected from the group consisting of lower alkyl, lower alkyloxy, hydroxyl; chloro, bromo, nitrile and trifluoromethyl;
Z is selected from the group consisting of lower alkyl, lower alkoxy, hydroxyl, fluoro, chloro, bromo, nitrile and trifluoromethyl;
m, n, and p are 0 or an integer of 1 or 2; and
A is (CR₂)ₐ and a is 1.

4. (canceled)

5. The compound of claim 3 wherein m and n are 0 and Z is selected from the group consisting of fluoro, chloro, bromo, and methyl.

6. (canceled)

7. (canceled)

9. (canceled)

10. (canceled)

11. The compound of claim 1 wherein X and Y are independently selected from the group consisting of methyl, nitrile, methyloxy, chloro and bromo; and Z is selected from the group consisting of fluoro, iodo, chloro, methyl and bromo.

12. (canceled)

13. The compound of claim 1 wherein X and Y are independently selected from the group consisting of lower alkyl, lower alkyloxy, hydroxyl, chloro, bromo, nitrile and trifluoromethyl; and
Z is selected from the group consisting of lower alkyl, lower alkoxy, hydroxyl, fluoro, chloro, bromo, nitrile and trifluoromethyl;
m, n, and p are 0 or an integer of 1 or 2; and
A is S.

14. The compound of claim 1 wherein X and Y are independently selected from the group consisting of lower alkyl, lower alkyloxy, hydroxyl, chloro, nitrile, trifluoromethyl and bromo; and Z is selected from the group consisting of lower alkyl, lower alkoxy, hydroxyl, fluoro, chloro, bromo, iodo, nitrile and trifluoromethyl and methyl;
m, n, and p are 0 or an integer of 1 or 2; and
A is O.

15. The compound of claim 14 selected from the group consisting of:
2-(4-chloro-3-methylphenoxy)-2-(4-chlorophenyl)-N-(5-chloropyridin-2-yl)acetamide hydrochloride;
N-(5-bromopyridin-2-yl)-2-(4-chlorophenyl)-2-(p-tolyloxy)acetamide hydrochloride;
2-(4-chlorophenyl)-N-(5-chloropyridin-2-yl)-2-(4-methoxyphenoxy)acetamide hydrochloride;
2-(4-bromophenoxy)-2-(4-chlorophenyl)-N-(5-fluoropyridin-2-yl)acetamide hydrochloride;
2-(4-bromophenoxy)-N-(5-bromopyridin-2-yl)-2-(4-chlorophenyl)acetamide hydrochloride;
2-(4-bromophenoxy)-2-(4-chlorophenyl)-N-(5-chloropyridin-2-yl)acetamide hydrochloride;
2-(4-chlorophenoxy)-2-(4-chlorophenyl)-N-(5-fluoropyridin-2-yl)acetamide hydrochloride;
N-(5-bromopyridin-2-yl)-2-(4-chlorophenoxy)-2-(4-chlorophenyl)acetamide hydrochloride; and
2-(4-chlorophenoxy)-2-(4-chlorophenyl)-N-(5-chloropyridin-2-yl)acetamide hydrochloride.

16. The compound of claim 13 selected from the group consisting of:
N-(5-bromopyridin-2-yl)-2-phenyl-2-(phenylthio)acetamide hydrochloride;
N-(5-bromopyridin-2-yl)-2-(4-chlorophenyl)-2-(4-methoxyphenylthio)acetamide hydrochloride;
2-(4-chlorophenyl)-N-(5-chloropyridin-2-yl)-2-(4-methoxyphenylthio)acetamide hydrochloride
2-(4-chlorophenyl)-N-(5-chloropyridin-2-yl)-2-(4-fluorophenylthio)acetamide hydrochloride;
N-(5-bromopyridin-2-yl)-2-(4-chlorophenyl)-2-(4-fluorophenylthio)acetamide hydrochloride;
2-(4-chlorophenyl)-N-(5-chloropyridin-2-yl)-2-(3,4-dimethylphenylthio)acetamide hydrochloride;
N-(5-bromopyridin-2-yl)-2-(4-chlorophenyl)-2-(p-tolylthio)acetamide hydrochloride;
2-(4-chlorophenyl)-N-(5-chloropyridin-2-yl)-2-(p-tolylthio)acetamide hydrochloride;
N-(5-bromopyridin-2-yl)-2-(3,4-dichlorophenylthio)-2-phenylacetamide;
N-(5-chloropyridin-2-yl)-2-(3,4-dichlorophenylthio)-2-phenylacetamide;
N-(5-bromopyridin-2-yl)-2-(3-chlorophenylthio)-2-phenylacetamide hydrochloride;
2-(3-chlorophenylthio)-N-(5-chloropyridin-2-yl)-2-phenylacetamide hydrochloride;
N-(5-bromopyridin-2-yl)-2-(4-chlorophenyl)-2-(4-chlorophenylthio)acetamide hydrochloride;
2-(4-chlorophenyl)-2-(4-chlorophenylthio)-N-(5-chloropyridin-2-yl)acetamide hydrochloride;
2-(4-bromophenylthio)-N-(5-chloropyridin-2-yl)-2-phenylacetamide;
2-(4-bromophenylthio)-N-(5-bromopyridin-2-yl)-2-phenylacetamide;
2-(4-chlorophenylthio)-N-(5-chloropyridin-2-yl)-2-(4-fluorophenyl)acetamide;
2-(4-chlorophenylthio)-N-(5-chloropyridin-2-yl)-2-phenylacetamide; and
N-(5-iodopyridin-2-yl)-2-phenyl-2-(phenylthio)acetamide.

17. The compound of claim 3 selected from the group consisting of:
N-(5-chloropyridin-2-yl)-2,3-diphenylpropanamide hydrochloride;
N-(5-bromopyridin-2-yl)-2,3-diphenylpropanamide hydrochlorideN-(5-bromopyridin-2-yl)-2,3-diphenylpropanamide hydrochloride;
N-(5-cyanopyridin-2-yl)-2,3-diphenylpropanamide hydrochloride;
N-(5-bromo-6-methylpyridin-2-yl)-2,3-diphenylpropanamide hydrochloride; and
N-(5-methylpyridin-2-yl)-2,3-diphenylpropanamide hydrochloride.

18. (canceled)

19. The method of claim 21, wherein the diseases and conditions of the eye are selected from the group consisting of glaucoma, dry eye and angiogenesis disorders and pulmonary edema.

20. (canceled)

21. A method of treating a disorder associated with sphingosine-1-phosphate receptor modulation, which comprises administering to a patient in need thereof a therapeutically effective amount of at least one compound of Formula I: 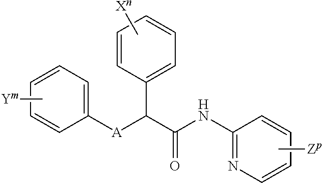
wherein:
A is O, S or (CR₂)ₐ wherein R is selected from the group consisting of H, or lower alkyl;
m, n, p are 0 or an integer of from 1 to 5;
a is 0 or 1,
X and Y are independently selected from the group consisting of alkyl, alkyloxy, hydroxyl, halogen, nitrile and trifluoromethyl;
Z is selected from the group consisting of alkyl, alkyloxy, hydroxyl, halogen, nitrile and trifluoromethyl; and
when a is 0;
X, Y are independently selected from the group consisting of alkyl, alkyloxy, hydroxyl, halogen, nitrile and trifluoromethyl;
Z is selected from the group consisting of alkyl, alkyloxy, hydroxyl, halogen, nitrile and trifluoromethyl;
provided however that when a is 0 and when the pyridyl ring is (a) 5-chloro pyridyl, then both (i) m and n are not 0 and (ii) X and Y are not chloro or methyl or (b) 5-bromo or iodo pyridyl, than both m and n are not 0.

22. A pharmaceutical composition comprising as active ingredient a therapeutically effective amount of a compound according to claim 1 and a pharmaceutically acceptable adjuvant, diluents or carrier.

23. A pharmaceutical composition according to claim 22 wherein the compound is selected from:
N-(5-cyanopyridin-2-yl)-2,3-diphenylpropanamide hydrochloride;
N-(5-methylpyridin-2-yl)-2,3-diphenylpropanamide hydrochloride;
N-(5-bromo-6-methylpyridin-2-yl)-2,3-diphenylpropanamide hydrochloride
N-(5-bromopyridin-2-yl)-2,3-diphenylpropanamide hydrochloride; and
N-(5-chloropyridin-2-yl)-2,3-diphenylpropanamide hydrochloride.

24. A pharmaceutical composition according to claim 22 wherein the compound is selected from:
N-(5-bromopyridin-2-yl)-2-phenyl-2-(phenylthio)acetamide hydrochloride;
N-(5-bromopyridin-2-yl)-2-(4-chlorophenyl)-2-(4-methoxyphenylthio)acetamide hydrochloride;
2-(4-chlorophenyl)-N-(5-chloropyridin-2-yl)-2-(4-methoxyphenylthio)acetamide hydrochloride;
2-(4-chlorophenyl)-N-(5-chloropyridin-2-yl)-2-(4-fluorophenylthio)acetamide hydrochloride;
N-(5-bromopyridin-2-yl)-2-(4-chlorophenyl)-2-(4-fluorophenylthio)acetamide hydrochloride;
2-(4-chlorophenyl)-N-(5-chloropyridin-2-yl)-2-(3,4-dimethylphenylthio)acetamide hydrochloride;
N-(5-bromopyridin-2-yl)-2-(4-chlorophenyl)-2-(p-tolylthio)acetamide hydrochloride;
2-(4-chlorophenyl)-N-(5-chloropyridin-2-yl)-2-(p-tolylthio)acetamide hydrochloride;
N-(5-bromopyridin-2-yl)-2-(3,4-dichlorophenylthio)-2-phenylacetamide;
N-(5-chloropyridin-2-yl)-2-(3,4-dichlorophenylthio)-2-phenylacetamide;
N-(5-bromopyridin-2-yl)-2-(3-chlorophenylthio)-2-phenylacetamide hydrochloride;
2-(3-chlorophenylthio)-N-(5-chloropyridin-2-yl)-2-phenylacetamide hydrochloride;
N-(5-bromopyridin-2-yl)-2-(4-chlorophenyl)-2-(4-chlorophenylthio)acetamide hydrochloride;
2-(4-chlorophenyl)-2-(4-chlorophenylthio)-N-(5-chloropyridin-2-yl)acetamide hydrochloride;
2-(4-bromophenylthio)-N-(5-chloropyridin-2-yl)-2-phenylacetamide;
2-(4-bromophenylthio)-N-(5-bromopyridin-2-yl)-2-phenylacetamide;
2-(4-chlorophenylthio)-N-(5-chloropyridin-2-yl)-2-(4-fluorophenyl)acetamide;
2-(4-chlorophenylthio)-N-(5-chloropyridin-2-yl)-2-phenylacetamide, and
N-(5-iodopyridin-2-yl)-2-phenyl-2-(phenylthio)acetamide.
